SEQUENCE LISTING (51 sequences)

SEQ ID NO: 1 - DNA, 1173 bp, Human
atgctgtcccgcaagggcatcatccccgaggaatacgtgctgacacgcctggcagaggaccctgccgagcccaggtaccgtgcccgccagcggagggcccgctttgtgtccaagaaaggcaactgcaacgtggcccacaagaacatccgggagcagggccgcttcctgcaggacgtgttcaccacgctggtggacctcaagtggccacacacattgctcatcttcaccatgtccttcctgtgcagctggctgctcttcgccatggcctggtggctcatcgccttcgcccacggtgacctggcccccagcgagggcactgctgagccctgtgtcaccagcatccactccttctcgtctgccttccttttctccattgaggtccaagtgactattggctttggggggcgcatggtgactgaggagtgcccactggccatcctgagcctcatcgtgcagaacatcgtggggctcatgatcaacgccatcatgcttggctgcatcttcatgaagactgcccaagcccaccgcagggctgagaccctcatcttcagcaagcatgcggtgatcgctctgcgccacggccgcctctgcttcatgctacgtgtgggtgacctccgcaagagcatgatcatcagcgccaccatccacatgcaggtggtacgcaagaccaccagccccgagggcgaggtggtgcccctccaccaggtggacatccccatggagaacggcgtgggtggcaacagcatcttcctggtggccccgctgatcatctaccatgtcattgatgccaacagcccactctacgacctggcacccagcgacctgcaccaccaccaggacctcgagatcatcgtcatcctggaaggcgtggtggaaaccacgggcatcaccacccaggcccgcacctcctacctggccgatgagatcctgtggggccagcgctttgtgcccattgtagctgaggaggacggacgttactctgtggactactccaagtttggcaacaccatcaaagtgcccacaccactctgcacggcccgccagcttgatgaggaccacagcctactggaagctctgaccctcgcctcagcccgcgggcccctgcgcaagcgcagcgtgcccatggccaaggccaagcccaagttcagcatctctccagattccctgtcctga

SEQ ID NO: 2 - protein, 390 aa, Human
MLSRKGIIPEEYVLTRLAEDPAEPRYRARQRRARFVSKKGNCNVAHKNIREQGRFLQDVFTTLVDLKWPHTLLIFTMSFLCSWLLFAMAWWLIAFAHGDLAPSEGTAEPCVTSIHSFSSAFLFSIEVQVTIGFGGRMVTEECPLAILILIVQNIVGLMINAIMLGCIFMKTAQAHRRAETLIFSKHAVIALRHGRLCFMLRVGDLRKSMIISATIHMQVVRKTTSPEGEVVPLHQVDIPMENGVGGNSIFLVAPLIIYHVIDANSPLYDLAPSDLHHHQDLEIIVILEGVVETTGITTQARTSYLADEILWGQRFVPIVAEEDGRYSVDYSKFGNTIKVPTPLCTARQLDEDHSLLEALTLASARGPLRKRSVPMAKAKPKFSISPDSLS

SEQ ID NO: 3 - DNA, 14 bp, Human
ctgggctcggcagg

SEQ ID NO: 4 - DNA, 14 bp, Human
ctgggctyggcagg

SEQ ID NO: 5 - DNA, 14 bp, Human
ctgggcttggcagg

SEQ ID NO: 6 - protein, 33 aa, Human
MLSRKGIIPEEYVLTRLAEDPAEPRYRARQRRA

SEQ ID NO: 7 - protein, 47 aa, Rat
MLSRKGIIPEEYVLTRLAEDPTEPRYRTRERPIVAEEDGRYSVDYSK

SEQ ID NO: 8 - protein, 47 aa, Mouse
MLSRKGIIPEEYVLTRLAEDPAEPRYRTRERPIVAEEDGRYSVDYSK

SEQ ID NO: 9 - protein, 47 aa, Guinea pig
MLSRKGIIPEEYVLTRLAEDPTEPRYRARERPIVAEEDGRYSVDYSK

SEQ ID NO: 10 - protein, 47 aa, Rabbit
MLSRKGIIPEEYVLTRLAEDPAEPRYRARERPIVAEEDGRYSVDYSK

SEQ ID NO: 11 - protein, 47 aa, American bullfrog; identical to SEQ ID NO: 10

SEQ ID NO: 12 - protein, 47 aa, Bovine; identical to SEQ ID NO: 10

SEQ ID NO: 13 - protein, 47 aa, Cat; identical to SEQ ID NO: 10

SEQ ID NO: 14 - protein, 47 aa, Chimpanzee
MLSRKGIIPEEYVLTRLAEDPAEPRYRARQRPIVAEEDGRYSVDYSK

SEQ ID NO: 15 - protein, 47 aa, Horse; identical to SEQ ID NO: 10

SEQ ID NO: 16 - protein, 47 aa, Giant panda; identical to SEQ ID NO: 9

SEQ ID NO: 17 - protein, 47 aa, African elephant
MLSRKGIIPEEYVLTRLAEDPAEPRYHARERPIVAEEDGRYSVDYSK

SEQ ID NO: 18 - protein, 47 aa, Polar bear; identical to SEQ ID NO: 10

SEQ ID NO: 19 - protein, 40 aa, Chinese liver fluke
MLSRKGIIPEEYVLTRLAEDPAEPPIVAEEDGRYSVDYSK

SEQ ID NO: 20 - protein, 47 aa, Zebrafish
MLSRKGLIPEDYLLTRLAEDVLQPKFKTKQRPTVSEEDGMYAVDYSK

SEQ ID NO: 21 - protein, 47 aa, Japanese pufferfish
MLSRKGLIPEEYLLTRLAEDVLQPKFKAKQGPTVSEEEGMYAVDYSK

SEQ ID NO: 22 - protein, 25 aa, Spotted green pufferfish
MPKFKAKQRPTVSEEEGAYAVDYSK

SEQ ID NO: 23 - protein, 47 aa, Channel catfish
MLSREGVIPDDYVLTRLAEDVLQPKFKTKQRPTVSEDDGMYAVDYSK

SEQ ID NO: 24 - protein, 19 aa, Human
YSKFGNTIKVPTPLCTARQ

SEQ ID NO: 25 - protein, 19 aa, Rat
YSKFGNTVKVPTPLCTARQ

SEQ ID NO: 26 - protein, 19 aa, Mouse
YSKFGNTIKVPTPLCTARQ

SEQ ID NO: 27 - protein, 19 aa, Rabbit
YSKFGNTVKVPTPLCTARQ

SEQ ID NO: 28 - protein, 19 aa, Bovine
YSKFGNTIKVPTPLCTARQ

SEQ ID NO: 29 - protein, 19 aa, Cat
YSKFGNTIKVPTPLCTARQ

SEQ ID NO: 30 - protein, 19 aa, Chimpanzee
YSKFGNTIKVPTPLCTARQ

SEQ ID NO: 31 - protein, 19 aa, Horse
YSKFGNTIKVPTPLCTARQ

SEQ ID NO: 32 - protein, 19 aa, African elephant
YSKFGNTIKVPTPLCTARQ

SEQ ID NO: 33 - protein, 19 aa, Polar bear
YSKFGNTIKVPTPLCTARQ

SEQ ID NO: 34 - protein, 19 aa, Zebrafish
YSKFGNTVKVATPNSSART

SEQ ID NO: 35 - protein, 19 aa, Channel catfish
YSKFGKTVKVPTPICSARK

SEQ ID NO: 36 - protein, 27 aa, Human
LCVRYENNLKPVLKHVKAYIKPGQKVG

SEQ ID NO: 37 - protein, 27 aa, Rat
LCVRYENNLKPVLKHVKAYIKPGQKVG

SEQ ID NO: 38 - protein, 27 aa, Mouse
LCVRYENNLKPVLKHVKAYIKPGQKVG

SEQ ID NO: 39 - protein, 27 aa, Rabbit
LCVRYENNLKPVLKHVKAYIKPGQKVG

SEQ ID NO: 40 - protein, 27 aa, Bovine
LCVRYENNLKPVLKHVKAYIKPGVKVG

SEQ ID NO: 41 - protein, 27 aa, Cat
LCVRYENNLKPVLKHVKAYIKPGQKVG

SEQ ID NO: 42 - protein, 27 aa, Chimpanzee
LCVRYENNLKPVLKHVKAYIKPGQKVG

SEQ ID NO: 43 - protein, 27 aa, Horse
LCVRYENNLKPVLKHVKAYIKPGQKVG

SEQ ID NO: 44 - protein, 27 aa, African elephant
LCVRYENNLKPVLKHVKAYIKPGQKVG

SEQ ID NO: 45 - protein, 27 aa, Polar bear
LCVRYENNLKPVLKHVKAYIKPGQKVG

SEQ ID NO: 46 - protein, 27 aa, Zebrafish
LCVRYDSMLKPVLKHVNAHINPGQKVG

SEQ ID NO: 47 - protein, 27 aa, Channel catfish
LCVRYDPMLKPALKHVNAFINPGEKVG

SEQ ID NO: 48 - protein, 27 aa, Human
LSVRYDSSLKPVLKHVNALIAPGQKIG

SEQ ID NO: 49 - DNA, 17 bp, artificial
  synthetic oligonucleotide primer
ccacgtccgaggggtgc

SEQ ID NO: 50 - DNA, 22 bp, artificial
  synthetic oligonucleotide primer
aggagtggatgctggtgacaca

SEQ ID NO: 51 - protein, 47 aa, Human; identical to SEQ ID NO: 14

(19) United States
(12) Patent Application Publication
(10) Pub. No.: US 2023/0398180 A1
(43) Pub. Date: Dec. 14, 2023

(54) METHODS AND MATERIALS FOR TREATING HEART FAILURE

(71) Applicant: Mayo Foundation for Medical Education and Research, Rochester, MN (US)

(72) Inventors: Andre Terzic, Rochester, MN (US); Atta Behfar, Rochester, MN (US)

(21) Appl. No.: 18/035,901

(22) PCT Filed: Nov. 10, 2021
(86) PCT No.: PCT/US2021/058699

§ 371 Date: May 8, 2023

Related U.S. Application Data
(60) Provisional application No. 63/111,916, filed on Nov. 10, 2020.

(51) Int. Cl. A61K 38/17; C12Q 1/6883

(52) U.S. Cl. CPC A61K 38/177; C12Q 1/6883; C12Q 2600/156; C12Q 2600/118; A61K 48/00

(57) ABSTRACT

This document provides methods and materials for identifying and/or treating a mammal (e.g., a human) having, or at risk of developing, heart failure (e.g., inherited heart failure). For example, methods and materials for identifying a mammal (e.g., a human) having, or at risk of developing, heart failure (e.g., inherited heart failure) by detecting the presence of a mutation in both copies of a KCNJ11 gene present in the mammal (e.g., a human) are provided. Methods and materials for treating a mammal (e.g., a human) identified as having, or as being at risk of developing, heart failure (e.g., inherited heart failure) also are provided.

### CROSS-REFERENCE TO RELATED APPLICATIONS

This application claims the benefit of U.S. Patent Application Ser. No. 63/111,916, filed on Nov. 10, 2020. The disclosure of the prior application is considered part of (and is incorporated by reference in) the disclosure of this application.

### TECHNICAL FIELD

This document relates to methods and materials for identifying and/or treating a mammal (e.g., a human) having, or at risk of developing, heart failure (e.g., inherited heart failure). For example, the presence of a mutation in both copies of a KCNJ11 gene present in a mammal (e.g., a human) can be used to identify the mammal as having, or as being at risk of developing, heart failure (e.g., inherited heart failure). In some cases, this document provides methods and materials for treating a mammal (e.g., a human) identified as having, or as being at risk of developing, heart failure (e.g., inherited heart failure). For example, nucleic acid designed to express a Kir6.2 polypeptide (or Kir6.2 polypeptides) can be administered to a mammal (e.g., a human) having, or at risk of developing, heart failure (e.g., inherited heart failure) to treat the mammal.

### BACKGROUND INFORMATION

The heart failure epidemic affects thirty million people worldwide (Ziaeian et al., Nat. Rev. Cardiol., 13:368-78 (2016)). Present in about two percent of adults, heart failure develops in one of five individuals in their lifetime (Benjamin et al., Circulation, 139:e1-e473 (2019)). Despite advanced management, heart failure remains a primary indication for recurrent hospitalization and causes death in two-thirds of patients within five years of diagnosis (Metra et al., Lancet, 390:1981-95 (2017)).

### SUMMARY

Myocardial ATP-sensitive potassium (K_{ATP}) channels include a tetrameric Kir6.2 pore complex including four polypeptide subunits encoded by KCNJ11, and a tetrameric SUR2A complex including four polypeptide subunits encoded by ABCC9 (Lorenz et al., J. Mol. Cell. Cardiol., 31:425-34 (1999)). Typically, a KCNJ11 gene present in a human contains a guanine (G) at nucleotide position 67 (e.g., as numbered in SEQ ID NO:1) and encodes a Kir6.2 polypeptide having a glutamic acid at amino acid position 23 (a Kir6.2-E23 polypeptide; e.g., as numbered in SEQ ID NO:2). Substitution of the G at nucleotide position 67 with an adenine (A) (e.g., a c.67G>A single nucleotide variant) in a KCNJ11 gene (e.g., to form a GAG triplet at nucleotide positions 67, 68, and 69) results in the nucleic acid sequence encoding a Kir6.2 polypeptide having a lysine at amino acid position 23 (a Kir6.2-E23K polypeptide). Homozygosity for a c.67G>A single nucleotide variant in KCNJ11, present in nearly a tenth of the population, has been associated with left ventricular dilation in hypertension and aberrant cardiac exercise response in cross-sectional studies and has been associated with left ventricular dilation in hypertension and aberrant cardiac exercise response in cross-sectional studies (Riedel et al., Hum. Genet., 116:133-45 (2005); Reyes et al., Hum. Genet., 123:665-7 (2008); and Reyes et al., Hum. Genet., 126:779-89 (2009)).

This document provides methods and materials for identifying and/or treating a mammal (e.g., a human) having, or at risk of developing, heart failure (e.g., inherited heart failure). For example, the presence of a mutation (e.g., a c.67G>A single nucleotide variant) in both copies of a KCNJ11 gene present in a mammal (e.g., a human) can be used to identify the mammal as having, or as being at risk of developing, heart failure (e.g., inherited heart failure). In some cases, this document provides methods and materials for treating a mammal (e.g., a human) identified as having, or as being at risk of developing, heart failure (e.g., inherited heart failure). For example, nucleic acid designed to express a Kir6.2-E23 polypeptide (or Kir6.2-E23 polypeptides) can be administered to a mammal (e.g., a human) having, or at risk of developing, heart failure (e.g., inherited heart failure) to treat the mammal.

A diploid mammal such as a human has two copies of each gene present in its genome. As described herein, the presence of a c.67G>A single nucleotide variant in both copies of a KCNJ11 gene present in a mammal (e.g., homozygosity for the c.67G>A single nucleotide variant) can be used to identify that mammal as having, or as being at risk for developing, heart failure. For example, homozygosity for the c.67G>A single nucleotide variant results in the presence of only Kir6.2-E23K variant polypeptides within the Kir6.2 pore complex of K_{ATP} channels (KK homozygotes) in the mammal. KK homozygotes can be at increased risk of developing hearth failure. Also as described herein, delivering nucleic acid designed to express a Kir6.2-E23 polypeptide to cardiac cells within a mammal can allow the cardiac cells to produce K_{ATP} channels having at least one Kir6.2-E23 polypeptide in a tetrameric Kir6.2 pore complex.

Having the ability to identify a mammal (e.g., a human) as having, or as being at risk of developing, heart failure provides a unique and unrealized opportunity to provide early intervention for that mammal and/or to reduce heart failure risk for that mammal. Further, having the ability to provide cardiac cells within a living mammal with Kir6.2-E23 polypeptides using the methods and materials described herein can allow clinicians and patients (e.g., humans identified as having a c.67G>A single nucleotide variant in both copies of a KCNJ11 gene present in a mammal) to create K_{ATP} channels having at least one Kir6.2-E23 polypeptide in a tetrameric Kir6.2 pore complex to slow, delay, or eliminate heart failure progression in the patient. Accordingly, the methods and materials provided herein have wide relevance for clinical practice and clinical research.

In general, one aspect of this document features methods for assessing a mammal. The methods can include, or consist essentially of, (a) detecting a presence or absence of a c.67G>A single nucleotide variant in both copies of a KCNJ11 gene present in a sample from a mammal; (b) classifying the mammal as being likely to develop heart failure if the presence of the c.67G>A single nucleotide variant is detected in both copies of the KCNJ11 gene; and (c) classifying the mammal as not being likely to develop heart failure if the absence of the mutation in both copies of said KCNJ11 gene is detected. The mammal can be a human. The method can include detecting the presence of the c.67G>A single nucleotide variant in both copies of a KCNJ11 gene. The method can include classifying the mammal as being likely to develop heart failure. The sample can include genomic DNA from the mammal. The detecting can include restriction enzyme digestion of the genomic DNA.

In another aspect, this document features methods for treating a mammal at risk of developing heart failure. The methods can include, or consist essentially of, administering, to cells within a mammal identified as having a c.67G>A single nucleotide variant in both copies of a KCNJ11 gene of the mammal, nucleic acid encoding a Kir6.2-E23 polypeptide, where the Kir6.2-E23 polypeptide is expressed by the cells, and where the cells form functional K_{ATP} channels (e.g., K_{ATP} channels having at least one Kir6.2-E23 polypeptide). The mammal can be a human. The cells can be cardiac cells. The Kir6.2-E23 polypeptide can be a human Kir6.2-E23 polypeptide. The nucleic acid encoding the Kir6.2-E23 polypeptide can be administered to the cells in the form of a viral vector. The viral vector can be an adeno-associated viral vector, a Sendai viral vector, a lentiviral vector, a retroviral vector, an adenoviral vector, or a herpes simplex viral vector. The nucleic acid encoding the Kir6.2-E23 polypeptide can be administered to the cells in the form of a non-viral vector. The non-viral vector can be selected an extracellular vesicle, a liposome, or an expression plasmid. The nucleic acid encoding the Kir6.2-E23 polypeptide can be operably linked to a promoter sequence. The administration of the nucleic acid encoding a Kir6.2-E23 polypeptide can include an intracoronary injection, an endomyocardial injection, an epicardial injection, a coronary sinus injection, or a pericardial injection. The method can be effective to delay onset of a symptom of heart failure. The symptom of heart failure can be shortness of breath, fatigue, weakness, swelling in the legs, ankles, and/or feet, rapid heartbeat, irregular heartbeat, reduced ability for activity, reduced ability to exercise, persistent cough or wheezing, increased need to urinate at night, lack of urine production, swelling of the abdomen, rapid weight gain, lack of appetite, nausea, difficulty concentrating, decreased alertness, sudden shortness of breath and coughing, severe shortness of breath and coughing, trouble sleeping when lying flat, or any combinations thereof.

In another aspect, this document features methods for treating a mammal having heart failure. The methods can include, or consist essentially of, administering, to cells within a mammal identified as having a c.67G>A single nucleotide variant in both copies of a KCNJ11 gene of the mammal, nucleic acid encoding a Kir6.2-E23 polypeptide, where the Kir6.2-E23 polypeptide is expressed by the cells, and where the cells form functional K_{ATP} channels (e.g., K_{ATP} channels having at least one Kir6.2-E23 polypeptide). The mammal can be a human. The cells can be cardiac cells. The Kir6.2-E23 polypeptide can be a human Kir6.2-E23 polypeptide. The nucleic acid encoding the Kir6.2-E23 polypeptide can be administered to the cells in the form of a viral vector. The viral vector can be an adeno-associated viral vector, a Sendai viral vector, a lentiviral vector, a retroviral vector, an adenoviral vector, or a herpes simplex viral vector. The nucleic acid encoding the Kir6.2-E23 polypeptide can be administered to the cells in the form of a non-viral vector. The non-viral vector can be an extracellular vesicle, a liposome, or an expression plasmid. The nucleic acid encoding the Kir6.2-E23 polypeptide can be operably linked to a promoter sequence. The administration of the nucleic acid encoding a Kir6.2-E23 polypeptide can include an intracoronary injection, an endomyocardial injection, an epicardial injection, a coronary sinus injection, or a pericardial injection. The method can be effective to reduce a symptom of heart failure. The symptom of heart failure can be shortness of breath, fatigue, weakness, swelling in the legs, ankles, and/or feet, rapid heartbeat, irregular heartbeat, reduced ability for activity, reduced ability to exercise, persistent cough or wheezing, increased need to urinate at night, lack of urine production, swelling of the abdomen, rapid weight gain, lack of appetite, nausea, difficulty concentrating, decreased alertness, sudden shortness of breath and coughing, severe shortness of breath and coughing, trouble sleeping when lying flat, or any combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Participant Flow Diagram. Unrelated (n=2042) men and women were enrolled and completed clinical examination with 2031 unique individuals genotyped for Kir6.2-E23K variants. Follow-up (up to 20.6 years) included a two-stage clinical re-examination (4.0±0.3 years) and medical record abstraction study design.

FIGS. 2A-2B. KCNJ11 Encoded Kir6.2-E23K and Heart Failure Risk. FIG. 2A shows heart failure event accrual for KK, EE and EK carriers in a community-based population during a 20-year follow-up presented as Kaplan-Meier curves. The number of individuals at risk by genotype is tabulated at 3-year intervals. FIG. 2B shows cardiovascular and heart failure (inset)-associated death across KK, EE and EK genotypes presented as Kaplan-Meier curves. The number of individuals at risk by genotype is tabulated at 3-year intervals.

FIGS. 3A-3B. Independent and Combined Heart Failure Risk. FIG. 3A shows age/sex-adjusted hazard ratios (95% confidence interval, CI) for developing heart failure (HF) in subjects with KK genotype, hypertension, diabetes or coronary artery disease. FIG. 3B shows age/sex-adjusted hazard ratios for developing heart failure (HF) in KK carriers also diagnosed with hypertension, diabetes or coronary artery disease. Individuals without the respective risk factor are used as reference.

FIG. 4. Heart Failure Accrual Following Myocardial Infarction. Kaplan-Meier curves depict incident heart failure for KK and EE/EK carriers post-myocardial infarction (MI). The number of individuals at risk by genotype is shown at 3-year intervals.

FIGS. 5A-5B. Derived Induced Pluripotent Stem Cells from Heart Failure-Free Donors. Engineered from donor fibroblasts, induced pluripotent stem (iPS) cells carry a nucleic acid sequence encoding the EE, EK, or KK variant (FIG. 5A; SEQ ID NOs:3, 4, and 5, respectively) with pluripotency markers detected by quantitative reverse transcription polymerase chain reaction (FIG. 5B).

FIGS. 6A-6D. Cardiomyocyte Behavior According to Genotype Status. Superimposed calcium transients (FIG. 6A left) and times of extrusion (FIG. 6A right) recorded from induced pluripotent stem cells derived cardiomyocytes of KK vs. EE/EK carriers. Oxygen consumption rate (OCR) and ratio of OCR to extracellular acidification rate (ECAR) in KK vs. EE/EK cardiomyocytes (FIG. 6B). Apoptosis in KK vs. EE/EK cardiomyocytes stained with TMR-red cell death (scale bars: 20 m, FIG. 6C). Under isoproterenol (2 μM) challenge, rate response in EE/EK vs. KK cardiomyocytes (FIG. 6D). Asterisk indicates a P value of less than 0.05.

FIGS. 7A-7C. Kir6.2-E23K and Structural Fidelity of the KATP Channel Complex. FIG. 7A zooms-in on the negatively charged E23 coupling upstream two adjacent Kir6.2 subunits (through attraction with the positively charged R325) and facilitating downstream Kir6.2-SUR2A communications (involving Kir6.2-K338 and SUR2A-E1319). Inset in FIG. 7A shows that the positively charged K23 variant would repulse R325 preventing subunit interactions. FIG. 7B maps the E23 neighborhood conforming to the resolved human K_{ATP} channel analog structure (PDB ID: 6C30). FIG. 7C depicts the Kir6.2 tetrameric structure (four individual subunits) assembled to form the K_{ATP} channel pore, surrounded by four regulatory sulfonylurea receptor (SUR) subunits (grey). E23 (black) maps to the outer-layer of each Kir6.2 subunit. Iterative Threading ASSEmbly Refinement (I-TASSER) atomic modeling program along with the Symmdock docking program were used to map K_{ATP} channel interactions.

FIG. 8. Homozygous KK status aggravates heart failure outcomes. Upper Panels: Long-term surveillance of heart failure patients exposes accelerated incidence in KK versus EE or EK carriers of cardiovascular readmissions or death (Left) or heart failure associated readmissions or death (Right). Lower Left Panel: Age/sex-adjusted hazard ratios (95% confidence interval, CI) for developing cardiovascular or heart failure associated readmissions or death in KK versus EE or EK carriers. Lower Right Panel: Cardiovascular and heart failure associated length of stay as a function of genetic variance. KK individuals required a prolonged length of stay for cardiovascular and heart failure related admissions. Inset, KK homozygotes experienced doubling of cardiovascular readmissions compared to EE/EK.

FIG. 9. KK is a risk for poor heart failure outcome. Upper Left Panel: Long-term surveillance of heart failure patients exposes accelerated incidence in KK versus EE or EK carriers of cardiovascular readmissions or death (Main) or heart failure associated readmissions or death (Inset). Upper Right Panel: Cardiovascular and heart failure associated length of stay as a function of genetic variance. KK individuals required a prolonged length of stay for cardiovascular and heart failure related admissions. Inset, Average length of stay per person shows extended stay for KK carriers. Table Inset, Median length of stay as a function of genetic variance. Lower Left Panel: Age/sex-adjusted hazard ratios (95% confidence interval, CI) for developing cardiovascular or heart failure associated readmissions or death in KK versus EE or EK carriers. Lower Right Panel: Equivalent ejection fraction in heart failure patients across genetic variance.

FIG. 10. KCNJ11 encoded Kir6.2-E23K (KK) and heart failure risk. Left panel shows heart failure event accrual in high-risk and low-risk subpopulations dependent on KK carrier status. High-risk and low-risk subpopulations are those with vs without hypertension, diabetes, coronary artery disease and/or elevated body mass index ≥30 Kg/m². Right panel shows heart failure event accrual in individuals with elevated and normal NT-proBNP dependent on KK carrier status. Baseline elevated NT-proBNP was defined as >125 pg/mL. Data points are from a community-based population (N=1961 individuals) during a 20-year follow-up presented as Kaplan-Meier curves. The number of individuals at risk by genotype is tabulated at 5-year intervals.

FIG. 11. Upper: Long-term surveillance of heart failure free individuals exposes accelerated heart failure accrual in high-risk individuals (those with hypertension, diabetes, coronary artery disease and/or elevated body mass index ≥30 Kg/m²) compared to those otherwise considered at low-risk. Middle: Long-term surveillance of heart failure free individuals exposes accelerated heart failure accrual in individuals with clinically elevated NT-pro BNP (>125 pg/mL) compared to those with NT-pro BNP within the normal range. Lower: Long-term surveillance of heart failure free individuals exposes accelerated heart failure accrual in KK compared to EE/EK carriers.

FIG. 12. Pooled Cohort Equation (PCE) for heart failure risk. Left panel shows original PCE model performance. Right panel shows PCE model performance with KK genotype consideration. Data points are from a community-based population (N=1961 individuals) during a 10-year follow-up presented as area under the curve plots.

FIG. 13. Improved Net Reclassification Index (NRI) for heart failure risk. KK genotype consideration improved Pooled Cohort Equation (PCE) model performance, reclassifying 8.2% of the population vulnerable to heart failure. Reclassification table where blue cells indicate subjects whose risk prediction improved under the PCE augmented model, and orange cells indicate subjects whose risk prediction worsened under the PCE augmented model (higher risk prediction is good for events and bad for non-events). Event NRI=up−#down/total events; Non-event NRI=#down−#up/total non-events; NRI=Event NR if non-event NRI. Data points are from a community-based population (N=1941 individuals) during a 10-year follow-up.

FIGS. 14A-14B. Genotype-phenotype relationships. FIG. 14A. Conventional one-to-one relationship adopts a gene-to-protein-to-pathophenotype. Heterozygote could be either dominant or recessive mutant, depending on phenotype outcomes. FIG. 14B. Random and independent co-assembly of EK heterozygotes produced 16 different Kir6.2 channel subtype options, grouped into 4E, 3E+4, 2E+2K, E+3K, and 4K. The distribution of 5 categorized groups is affected by allele frequency of E and K, and community-cohort allele frequency produced asymmetric outcomes of subtype channels, a departure from the typical normal distribution.

FIG. 15. Therapeutic modalities including both viral and non-viral gene therapy approaches and gene-editing technology enable the transformation of the vulnerable K into resilient E.

FIGS. 16A-16B. Bioinformatics assessment of human Kir6.2 E23K polymorphism. FIG. 16A. Single nucleotide polymorphism surveillance programs predicted a moderate pathophenotype generated by the E23K missense mutation. FIG. 16B. The E23-containing amino terminus constituted an intrinsically disordered protein region within Kir6.2 (SEQ ID NO:6).

FIG. 17A-17D. Two molecular structures of human Kir6.2 at monomer and homomer levels encompassing E23. FIG. 17A. Within an alpha-helix and loop Kir6.2 monomer representation, the negatively charged E23 residue, along with surrounding charged residues, occupied a locale at the interface between transmembrane and intracellular regions. FIG. 17B. Surface representation of a Kir6.2 tetramer (individual subunits), viewed from the extracellular side, revealed the E23 mapped at the periphery of the assembled pore structure. FIG. 17C. The negatively charged E23 interacts with the positively charged R325 counterpart residing within a neighboring Kir6.2 subunit to form a permissive electrostatic interaction. FIG. 17D. E23/R325 form an attractive force pairing, visualized from the extracellular side (following clipping of the front and back) of the Kir6.2 structure.

FIG. 18. Sequence alignments of KCNJ11-encoded Kir6.2 orthologs (SEQ ID NOs: 51, 7, 8, 9, 10, 11, 12, 13, 14, 15, 16, 17, 18, 19, 20, 21, 22, and 23, as numbered from top to bottom) revealed evolutionarily conserved amino acids forming ionic pair interactions. The E23/R325 pair was highly conserved maintaining electrostatic interactions across species. If the E23 equivalent residue was an uncharged amino acid, the R325 counterpart was also uncharged to sustain local conformation. Accession numbers of UniProt Knowledgebase, the central hub for the collection of functional information on proteins, are included.

FIGS. 19A-19C. K_{ATP} channel pore (Kir6.2) and regulatory (SUR) inter-subunit interaction. FIG. 19A. Putative Kir6.2/SUR interacting residues between Kir6.2 K338 (SEQ ID NOs: 24, 25, 26, 27, 28, 29, 30, 31, 32, 33, 34, and 35, as numbered from top to bottom) and SUR2A E1318 (SEQ ID NOs: 36, 37, 38, 39, 40, 41, 42, 43, 44, 45, 46, and 47, as numbered from top to bottom) or SUR1 (SEQ ID NO:48) were highly conserved ensuring discernible ionic interaction. FIG. 19B. Restraint-enforced molecular docking delineated the overall Kir6.2-SUR architecture where the cytoplasmic locale of Kir6.2 was intimately positioned in the SUR proximity. FIG. 19C. Close-up of the docked structure revealed downstream salt-bridge pairing between Kir6.2 and the SUR subunit-bound nucleotide conformation (human Kir6.2 and SUR1 sequence). Kir6.2 E23K polymorphism destabilized the Kir6.2-Kir6.2 interaction, and the subsequent Kir6.2-SUR communication.

FIGS. 20A-20B. Five E23K polymorphism produced a non-permissive scenario. FIG. 20A. The E23K missense mutation indices a switch from the negatively charged glutamic acid (E) to a positively charged lysine (K) residue. FIG. 20B. The positively charged K23 variant is disruptive to the electrostatic pairing normally occurring between E23 and R325.

FIGS. 21A-21E. Six molecular dynamics simulations in lipid bilayer documented the criticality of E23 for permissive interaction, interrupted by K23. FIG. 21A. Cα root mean square deviation of E23- versus K23-containing Kir6.2 tetramers displayed conformational drift during prolonged simulation. FIG. 21B. K23/R325 pair could not be in close contact for H-bonding and salt bridge interaction, whereas E23/R325 was accommodative during hundreds nanosecond-long dynamics simulations. FIG. 21C The E23/R325 pair displays a lesser fluctuating distance than K23/R325. FIG. 21D and FIG. 21E End-of-simulation molecular structure, embedded in lipid bilayer, captures E23/R325 paired, contrasting K23 unpaired from R325.

FIG. 22. Organ failure in KK homozygous hearts. Left panel: While pre-stress (DOCA (−)) heart weight was equivalent between EK and KK genotypes, KK hearts demonstrated pathological weight increase under pro-hypertensive stress (DOCA (+)). Right panel: Under DOCA stress, EK hearts maintained contractility with wild mild cardiomegaly (top, echocardiography; bottom, macroscopic examination), indicating adaption to stress. In contrast, KK hearts developed, during the same follow-up period, maladaptive cardiomyopathy with severe chamber dilatation and reduced contractility (top), associated with pathological advanced cardiomegaly (bottom).

FIG. 23. ¹H NMR spectroscopy-based metabolic analysis of heart (KK versus EE). BCAAs (dark red) and other metabolites (black).

FIG. 24. Nucleic acid sequence that can encode a representative Kir6.2-E23 polypeptide (SEQ ID NO:1). The G at nucleotide position 67 is bolded, and the GAG triplet at nucleotide positions 67, 68, and 69 is italicized.

FIG. 25. Amino acid sequence of a representative Kir6.2-E23 polypeptide (SEQ ID NO:2). The E at amino acid position 23 bolded.

### DETAILED DESCRIPTION

This document provides methods and materials for identifying and/or treating a mammal (e.g., a human) having, or at risk of developing, heart failure (e.g., inherited heart failure). For example, the presence of a mutation (e.g., a c.67G>A single nucleotide variant) in both copies of a KCNJ11 gene present in a mammal (e.g., a human) can be used to identify the mammal as having, or as being at risk of developing, heart failure (e.g., inherited heart failure). In some cases, this document provides methods and materials for treating a mammal (e.g., a human) identified as having, or as being at risk of developing, heart failure (e.g., inherited heart failure). For example, nucleic acid designed to express a Kir6.2-E23 polypeptide (or Kir6.2-E23 polypeptides) can be administered to a mammal (e.g., a human) having, or at risk of developing, heart failure (e.g., inherited heart failure) to treat the mammal. As described herein, the presence of a c.67G>A single nucleotide variant in both copies of a KCNJ11 gene present in a mammal (e.g., homozygosity for the c.67G>A single nucleotide variant) can be used to identify that mammal as having, or as being at risk for developing, heart failure. In some cases, a mammal (e.g., a human) can be identified as having, or at risk of developing, heart failure (e.g., inherited heart failure) based, at least in part, on the presence of a mutation (e.g., c.67G>A single nucleotide variant) in both copies of a KCNJ11 gene present in a mammal (e.g., a human) in a sample obtained from the mammal. In some cases, this document provides methods and materials for treating a mammal (e.g., a human) identified as having, or as being at risk of developing, heart failure (e.g., inherited heart failure). For example, nucleic acid designed to express a Kir6.2-E23 polypeptide (or Kir6.2-E23 polypeptides) can be administered to a mammal (e.g., a human) identified as having, or at risk of developing, heart failure (e.g., inherited heart failure) to reduce the risk of that mammal developing heart failure.

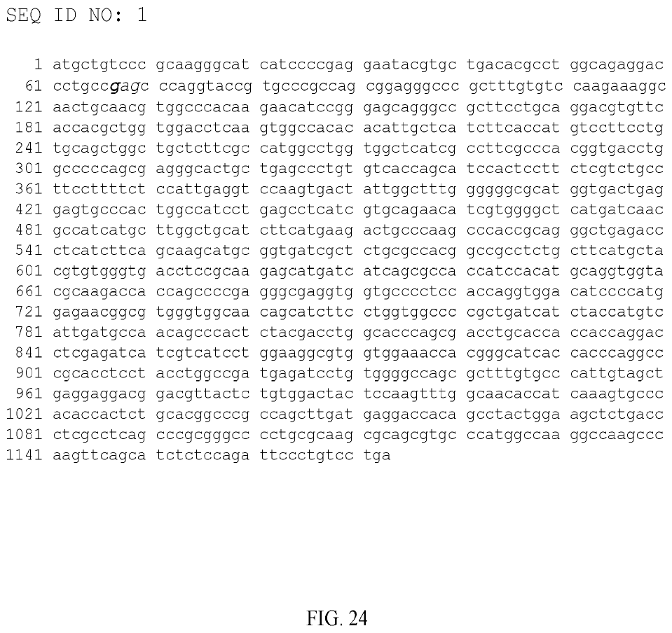

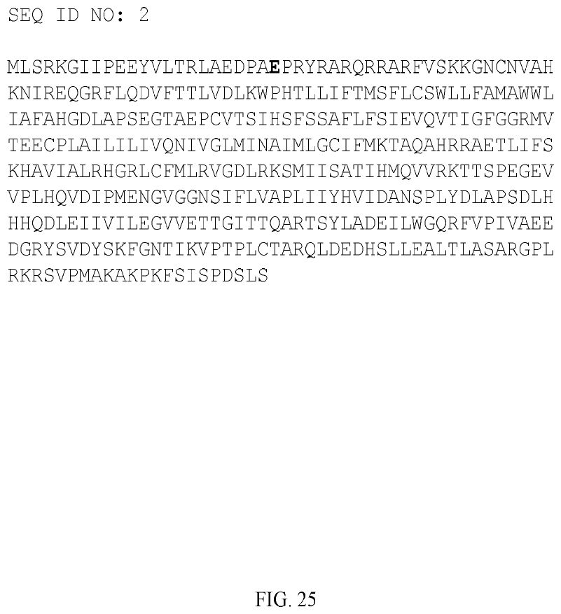

In some cases, a mammal (e.g., a human) can be identified as having, or as being at risk of developing, heart failure (e.g., inherited heart failure) based, at least in part, on the presence or absence of a mutation (e.g., c.67G>A single nucleotide variant) in both copies of a KCNJ11 gene present in a mammal (e.g., a human) in a sample (e.g., a sample containing one or more cells) obtained from the mammal. The term “mutation” as used herein with respect to nucleic acid refers to a modification in the nucleic acid sequence as compared to a wild type nucleic acid for a particular species. A mutation can be any type of mutation including, without limitation, an insertion of one or more nucleotides, a deletion of one or more nucleotides, an insertion of one or more nucleotides in combination with a deletion of one or more nucleotides (an INDEL), a substitution of one or more nucleotides (e.g., single nucleotide variant), and combinations thereof. In some cases, a mutation can be a single nucleotide variant (e.g., c.67G>A single nucleotide variant). In some cases, a mutation can be as compared to a wild type human KCNJ11 gene. In some cases, a mutation can be as compared to a wild type murine KCNJ11 gene. In some cases, a mutation can be as compared to a wild type primate KCNJ11 gene (e.g., a wild type Rhesus monkey KCNJ11 gene). Examples of wild type KCNJ11 genes include, without limitation, those genes having a nucleic acid sequence set forth in the National Center for Biotechnology Information (NCBI) database under GenBank® accession no. BC112358 (version BC112358.1), Gene ID no. 16514, and accession no. BV447782 (version BV447782.1). For example, a mutation can be as compared to the nucleic acid sequence set forth in SEQ ID NO:1 (see, e.g., FIG. 24). In some cases, a mutation in a nucleic acid sequence in a sample from a mammal can be as described in Example 1.

Any appropriate method can be used to detect the presence or absence of one or more mutations (e.g., c.67G>A single nucleotide variant) in a nucleic acid within a sample (e.g., a sample containing one or more cells) obtained from a mammal (e.g., a human). For example, sequencing (e.g., PCR-based sequencing), DNA hybridization, restriction enzyme digestion methods, and chromosomal microarray can be used to identify the presence or absence of one or more mutations (e.g., c.67G>A single nucleotide variant) in a nucleic acid. In some cases, the presence or absence of one or more mutations (e.g., c.67G>A single nucleotide variant) a nucleic acid within a sample from a mammal can be determined as described in Example 1.

Any appropriate mammal can be assessed and/or treated as described herein. Examples of mammals that can be assessed and/or treated as described herein include, without limitation, humans, non-human primates such as monkeys, dogs, cats, horses, cows, pigs, sheep, llamas, mice, rats, guinea pigs, rabbits, and hamsters.

Any appropriate sample from a mammal (e.g., a human) can be assessed as described herein (e.g., for the presence or absence of one or more mutations (e.g., c.67G>A single nucleotide variant) in a nucleic acid). In some cases, a sample can be a biological sample. In some cases, a sample can contain one or more biological molecules (e.g., nucleic acids such as DNA (genomic DNA) and RNA, polypeptides, carbohydrates, lipids, hormones, and/or metabolites). Examples of samples that can be assessed as described herein include, without limitation, fluid samples (e.g., whole blood, serum, plasma, urine, saliva, sputum, cerebrospinal fluid, and semen) and tissue samples (e.g., tissue samples obtained by biopsy) such as skin fibroblasts and myocardial tissue. Abiological sample can be a fresh sample or a fixed sample (e.g., a formaldehyde-fixed sample or a formalin-fixed sample). In some cases, a biological sample can be a processed sample (e.g., to isolate or extract one or more biological molecules). For example, a blood (e.g., plasma) sample can be obtained from a mammal (e.g., a human) and can be assessed for the presence or absence of one or more mutations (e.g., c.67G>A single nucleotide variant) in a nucleic acid to determine if the mammal has, or is at risk of developing, heart failure (e.g., inherited heart failure) based, at least in part, on the presence of a mutation (e.g., c.67G>A single nucleotide variant) in both copies of a KCNJ11 gene in the sample.

In some cases, a mammal can be identified as having, or as being at risk of developing, heart failure using any appropriate heart failure diagnostic technique. For example, medical history (e.g., the presence of congestive symptoms such as paroxysmal nocturnal dyspnea and bendopnea and/or functional limitations such as those as described in the New York Heart Association Functional Classifications), risk factors (e.g., high blood pressure, coronary artery disease, and diabetes), physical examination (e.g., for vital signs, evaluation of jugular venous pressure, pulmonary auscultation for signs of lung congestion, cardiac auscultation for abnormal heart sounds, and for signs of reduced peripheral circulation such as fluid buildup in your abdomen and legs (e.g., pitting edema)), blood tests (e.g., serological testing for natriuretic peptides and/or troponins such as N-terminal pro-B-type natriuretic peptide (NT-proBNP)), electrocardiography (ECG), and/or imaging tests (e.g., chest X-rays, cardiac computerized tomography (CT) scans, magnetic resonance imaging (MRI)), echocardiograms, coronary angiograms, and nuclear imaging) can be used to diagnose a human as having, or as being at risk of developing, heart failure.

This document also provides methods for treating a mammal (e.g., a human) identified as having, or as being at risk of developing, heart failure (e.g., inherited heart failure). As described herein, delivering nucleic acid designed to express a Kir6.2-E23 polypeptide to cardiac cells within a mammal can allow the cardiac cells to produce K_{ATP} channels having at least one Kir6.2-E23 polypeptide in a tetrameric Kir6.2 pore complex. In some cases, nucleic acid designed to express a Kir6.2-E23 polypeptide (or Kir6.2-E23 polypeptides) can be administered to a mammal (e.g., a human) identified as having, or at risk of developing, heart failure (e.g., inherited heart failure) based, at least in part, on the presence of a mutation (e.g., c.67G>A single nucleotide variant) in both copies of a KCNJ11 gene present in a mammal (e.g., a human) in a sample obtained from the mammal. For example, nucleic acid designed to express a Kir6.2-E23 polypeptide (or Kir6.2-E23 polypeptides) can be administered to a mammal (e.g., a human) identified as having, or at risk of developing, heart failure (e.g., inherited heart failure) to reduce the risk of that mammal developing heart failure.

Examples of Kir6.2-E23 polypeptides include, without limitation, those polypeptides having the amino acid sequence set forth in the UniProt Knowledgebase (UniProtKB) under accession no. Q14654 (isoform Q14654-1), accession no. Q8CCI6, and accession no. H2R5J9.

Any appropriate method can be used to deliver nucleic acid designed to express a Kir6.2-E23 polypeptide to cells within a living mammal. For example, nucleic acid encoding a Kir6.2-E23 polypeptide can be administered to a mammal using one or more viral vectors. For example, nucleic acid encoding a Kir6.2-E23 polypeptide can be administered to a mammal using one or more non-viral vectors.

In some cases, vectors for administering nucleic acid (e.g., nucleic acid designed to express a Kir6.2-E23 polypeptide) to cells can be used for transient expression of a Kir6.2-E23 polypeptide.

In some cases, vectors for administering nucleic acid (e.g., nucleic acid designed to express a Kir6.2-E23 polypeptide) to cells can be used for stable expression of a Kir6.2-E23 polypeptide. In cases where a vector for administering nucleic acid can be used for stable expression of a Kir6.2-E23 polypeptide, the vector can be engineered to integrate nucleic acid designed to express a Kir6.2-E23 polypeptide into the genome of a cell. In some cases, a vector can be engineered to integrate nucleic acid designed to express a Kir6.2-E23 polypeptide into the genome of a cell using any appropriate method. For example, gene therapy techniques can be used to integrate nucleic acid designed to express a Kir6.2-E23 polypeptide into the genome of a cell.

Vectors for administering nucleic acids (e.g., nucleic acid encoding a Kir6.2-E23 polypeptide) to cells can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, NJ (2002) and Viral Vectors for Gene Therapy: Methods and Protocols, edited by Curtis A. Machida, Humana Press, Totowa, NJ (2003).

When a vector used to deliver nucleic acid encoding a Kir6.2-E23 polypeptide to cells within a living mammal is a viral vector, any appropriate viral vector can be used. In some cases, a virus based vector can be derived from a positive-strand RNA virus. In some cases, a virus-based vector can be a chimeric viral vectors. Examples of viruses that virus-based vectors that can be used to deliver nucleic acid encoding a Kir6.2-E23 polypeptide to cells within a living mammal can be derived from include, without limitation, adenoviruses, adeno-associated viruses (AAVs), Sendai viruses, retroviruses, lentiviruses, and herpes simplex viruses. In some cases, nucleic acid encoding a Kir6.2-E23 polypeptide can be delivered to cells using AAV vectors (e.g., an AAV serotype 2 viral vector, an AAV serotype 5 viral vector, an AAV serotype 9 viral vector, or a recombinant AAV serotype viral vector such as an AAV serotype 2/5 viral vector), Sendai viral vectors, lentiviral vectors, retroviral vectors, adenoviral vectors, and herpes simplex viral vectors.

When a vector used to deliver nucleic acid encoding a Kir6.2-E23 polypeptide to cells within a living mammal is a non-viral vector, any appropriate non-viral vector can be used. In some cases, a non-viral vector can be an extracellular vesicle (e.g., exosome). In some cases, a non-viral vector can be a liposome (e.g., nano-liposomes). In some cases, a non-viral vector can be an expression plasmid (e.g., a cDNA expression vector).

In some cases, nucleic acid encoding a Kir6.2-E23 polypeptide can be administered to a mammal by direct injection of naked nucleic acid molecules.

In some cases, nucleic acid encoding a Kir6.2-E23 polypeptide can be administered to a mammal by direct injection of nucleic acid molecules complexed with lipids (e.g., nano-liposome complexes), polymers, or nanospheres.

In addition to nucleic acid encoding a Kir6.2-E23 polypeptide, a vector (e.g., a viral vector or a non-viral vector) can contain regulatory elements operably linked to the nucleic acid encoding a Kir6.2-E23 polypeptide. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a vector depends on several factors, including, without limitation, inducibility, targeting, and the level of expression desired. For example, a promoter can be included in a vector to facilitate transcription of a nucleic acid encoding a Kir6.2-E23 polypeptide. A promoter can be constitutive or inducible (e.g., in the presence of tetracycline), and can affect the expression of a nucleic acid encoding a polypeptide in a general or tissue-specific manner (e.g., cardiac specific promoters and muscle specific promoters). Examples of promoters that can be used to drive expression of a Kir6.2-E23 polypeptide in cells include, without limitation, cardiac α-myosin heavy chain promoters, cardiac myosin light chain-2 promoters, and cardiac troponin C promoters. As used herein, “operably linked” refers to positioning of a regulatory element in a vector relative to a nucleic acid in such a way as to permit or facilitate expression of the encoded polypeptide. For example, a vector can contain a promoter and nucleic acid encoding a Kir6.2-E23 polypeptide. In this case, the promoter is operably linked to a nucleic acid encoding a Kir6.2-E23 polypeptide such that it drives transcription in cells.

Nucleic acid encoding a Kir6.2-E23 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a Kir6.2-E23 polypeptide.

In some cases, Kir6.2-E23 polypeptides can be administered in addition to or in place of nucleic acid designed to express a Kir6.2-E23 polypeptide. For example, Kir6.2-E23 polypeptides can be delivered to cells within a mammal to allow the cells to produce K_{ATP} channels having at least one Kir6.2-E23 polypeptide in a tetrameric Kir6.2 pore complex.

Nucleic acid designed to express a Kir6.2-E23 polypeptide (or Kir6.2-E23 polypeptides) can be delivered to cells within a mammal via pericardial injection (e.g., direct injection into the pericardium), intramyocardial injection (e.g., direct injection into the myocardium, for example, as an endomyocardial injection or an epicardial injection), intracoronary injection (e.g., direct injection into the coronary vessels (e.g., arterial coronary vessels or venous coronary vessels) or sinus), intramuscular injection, intraperitoneal administration, intravenous administration, or oral delivery in nanoparticles and/or drug tablets, capsules, or pills.

In some cases, nucleic acid designed to express a Kir6.2-E23 polypeptide can express a Kir6.2-E23 polypeptide having the amino acid sequence set forth in in SEQ ID NO:2. For example, nucleic acid designed to express a polypeptide having the amino acid sequence set forth in in SEQ ID NO:2 can be administered to a mammal (e.g., a human) having, or at risk of developing, heart failure as described herein and used to treat the mammal.

In some cases, nucleic acid designed to express a Kir6.2-E23 polypeptide can express a variant of a Kir6.2-E23 polypeptide having the amino acid sequence set forth in in SEQ ID NO:2. For example, nucleic acid designed to express a polypeptide having the amino acid sequence set forth in in SEQ ID NO:2 can be administered to a mammal (e.g., a human) having, or at risk of developing, heart failure as described herein and used to treat the mammal. For example, a variant of a Kir6.2-E23 polypeptide can comprise or consist essentially of an amino acid sequence set forth in SEQ ID NO:2, provided that the amino acid sequence maintains an E at amino acid position 23, with one or more (e.g., one, two, three, four, five, six, seven, eight, nine, ten, or more) amino acid deletions, additions, substitutions, or combinations thereof. For example, nucleic acid designed to express a polypeptide comprising or consisting essentially of the amino acid sequence set forth in SEQ ID NO:2 can be administered to a mammal (e.g., a human) having, or at risk of developing, heart failure as described herein and used to treat the mammal.

Any appropriate amino acid residue set forth in SEQ ID NO:2 can be deleted, and any appropriate amino acid residue (e.g., any of the 20 conventional amino acid residues or any other type of amino acid such as ornithine or citrulline) can be added to or substituted within the sequence set forth in SEQ ID NO:2. The majority of naturally occurring amino acids are L-amino acids, and naturally occurring polypeptides are largely comprised of L-amino acids. D-amino acids are the enantiomers of L-amino acids. In some cases, a polypeptide provided herein can contain one or more D-amino acids. In some embodiments, a polypeptide can contain chemical structures such as F-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO:2 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:2 are set forth in the Table below.

Examples of conservative amino acid substitutions.

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, polypeptides can be designed to include the amino acid sequence set forth in SEQ ID NO:2 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

In some cases, a polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:2, provided that it includes at least one difference (e.g., at least one amino acid addition, deletion, or substitution) with respect to SEQ ID NO:2, can be used. For example, nucleic acid designed to express a polypeptide containing an amino acid sequence with between 90% and 99% sequence identity to the amino acid sequence set forth in SEQ ID NO:2 (or Kir6.2-E23 polypeptides themselves) can be designed and administered to a human having, or at risk of developing, heart failure, to treat the mammal.

Percent sequence identity is calculated by determining the number of matched positions in aligned amino acid sequences, dividing the number of matched positions by the length of an aligned amino acid sequence, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:2) is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:2), followed by multiplying the resulting value by 100. For example, an amino acid sequence that has 340 matches when aligned with the sequence set forth in SEQ ID NO:2 is 95.5 percent identical to the sequence set forth in SEQ ID NO:2 (i.e., 340÷356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In some cases, one or more gene therapy (e.g., gene replacement or gene editing) techniques can be administered to a mammal (e.g., a human) identified as having, or at risk of developing, heart failure (e.g., inherited heart failure) based, at least in part, on the presence of a mutation (e.g., c.67G>A single nucleotide variant) in both copies of a KCNJ11 gene present in a mammal (e.g., a human) to treat the mammal. For example, gene therapy components (e.g., gene editing components) designed to edit a c.67G>A single nucleotide variant present in a KCNJ11 gene can be delivered to a cell within a mammal (e.g., a human) having a c.67G>A single nucleotide variant in both copies of a KCNJ11 gene to convert at least one copy of the KCNJ11 gene to have a G at nucleotide position 67. Gene therapy components (e.g., gene editing components) designed to edit a mutation (e.g., c.67G>A single nucleotide variant) present in a KCNJ11 gene as described herein can be any appropriate gene therapy components. In some cases, a gene therapy component can be a nucleic acid (e.g., a targeting sequence and a donor nucleic acid). In some cases, a gene therapy component can be polypeptide (e.g., a nuclease). In some cases, a KCNJ11 gene edited as described herein can encode a Kir6.2-E23 polypeptide. When gene therapy components include a donor nucleic acid, the donor nucleic acid can be integrated into the genome (e.g., integrated in-frame into one or both KCNJ11 genes present in the mammal), and can encode a Kir6.2-E23 polypeptide.

Any appropriate gene therapy technique can be used to edit a mutation (e.g., a c.67G>A single nucleotide variant) present in a KCNJ11 gene in a cell within a mammal (e.g., a human having, or at risk of developing, heart failure based, at least in part, on the presence of a mutation (e.g., c.67G>A single nucleotide variant) in both copies of a KCNJ11 gene present in the human). Examples of gene therapy techniques that can be used to edit a mutation (e.g., a c.67G>A single nucleotide variant) present in a KCNJ11 gene in a cell within a mammal include, without limitation, gene replacement (e.g., using homologous recombination or homology-directed repair), gene editing, base editing, and prime editing.

In some cases, clustered regularly interspaced short palindromic repeat (CRISPR)/CRISPR-associated (Cas) nuclease (CRISPR/Cas) gene editing (e.g., therapeutic gene editing) techniques can be used to edit a mutation (e.g., a c.67G>A single nucleotide variant) present in a KCNJ11 gene in a cell within a mammal (e.g., a human having, or at risk of developing, heart failure based, at least in part, on the presence of a mutation (e.g., c.67G>A single nucleotide variant) in both copies of a KCNJ11 gene present in the human). CRISPR/Cas molecules are components of a prokaryotic adaptive immune system that is functionally analogous to eukaryotic RNA interference, using RNA base pairing to direct nucleic acid cleavage resulting in double stranded breaks (DSBs) about 3-4 nucleotides upstream of a protospacer adjacent motif (PAM) sequence (e.g., NGG). Directing nucleic acid DSBs with the CRISPR/Cas system requires two components: a Cas nuclease, and a guide RNA (gRNA) targeting sequence directing the Cas to cleave a target DNA sequence (Makarova et al., Nat Rev Microbiol, 9(6):467-477 (2011); and Jinek et al., Science, 337(6096):816-821 (2012)). The CRISPR/Cas system can be used in bacteria, yeast, humans, and zebrafish, as described elsewhere (see, e.g., Jiang et al., Nat Biotechnol, 31(3):233-239 (2013); Dicarlo et al., Nucleic Acids Res, doi:10.1093/nar/gkt135, 2013; Cong et al., Science, 339(6121):819-823 (2013); Mali et al., Science, 339(6121):823-826 (2013); Cho et al., Nat Biotechnol, 31(3):230-232 (2013); and Hwang et al., Nat Biotechnol, 31(3):227-229 (2013)). A CRISPR/Cas system can include any appropriate Cas nuclease. Cas nucleases can be as described elsewhere (see, e.g., Shalem et al., 2014 Science 343:84-87; and Sanjana et al., 2014 Nature methods 11: 783-784).

In some cases, a TALEN system can be used (e.g., can be introduced into one or more glial cells) to edit a mutation (e.g., a c.67G>A single nucleotide variant) present in a KCNJ11 gene in a cell within a mammal (e.g., a human having, or at risk of developing, heart failure based, at least in part, on the presence of a mutation (e.g., c.67G>A single nucleotide variant) in both copies of a KCNJ11 gene present in the human). Transcription activator-like (TAL) effectors are found in plant pathogenic bacteria of the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al., Science 318:648-651, 2007; Sugio et al., Proc Natl Acad Sci USA 104:10720-10725, 2007; and Romer et al., Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site selection and engineering of new TALENs with binding specificity for the selected sites. For example, an engineered TAL effector DNA binding domain targeting sequence can be fused to a nuclease to create a TALEN that can create nucleic acid DSBs at or near the sequence targeted by the TAL effector DNA binding domain. Directing nucleic acid DSBs with the TALEN system requires two components: a nuclease, and TAL effector DNA-binding domain directing the nuclease to a target DNA sequence (see, e.g., Schornack et al., J. Plant Physiol. 163:256, 2006). A TALEN system can include any appropriate nuclease. In some cases, a nuclease can be a non-specific nuclease. In some cases, a nuclease can function as a dimer. For example, when a nuclease that functions as a dimer is used, a highly site-specific restriction enzyme can be created. For example, each nuclease monomer can be fused to a TAL effector sequence that recognizes a different DNA target sequence, and only when the two recognition sites are in close proximity do the inactive monomers come together to create a functional enzyme. Examples of nucleases that can used in a TALEN system described herein include, without limitation, FokI, HhaI, HindIII, NotI, BbvCI, EcoRI, BglI, and AwI. For example, a nuclease of a TALEN system can include a FokI nuclease (see, e.g., Kim et al. (1996) Proc. Natl. Acad. Sci. USA 93:1156-1160).

In some cases, the methods and materials described herein can be used as the sole active agent used to treat a mammal having, or at risk of developing, heart failure (e.g., a human having, or at risk of developing, heart failure based, at least in part, on the presence of a mutation (e.g., c.67G>A single nucleotide variant) in both copies of a KCNJ11 gene present in the human). For example, nucleic acid encoding a Kir6.2-E23 polypeptide (or Kir6.2-E23 polypeptides) can be used as the sole active agent used to treat a mammal having, or at risk of developing, heart failure (e.g., a human having, or at risk of developing, heart failure based, at least in part, on the presence of a mutation (e.g., c.67G>A single nucleotide variant) in both copies of a KCNJ11 gene present in the human).

In some cases, the methods and materials described herein can include one or more (e.g., one, two, three, four, five or more) additional therapeutic agents used to treat a mammal having, or at risk of developing, heart failure (e.g., a human having, or at risk of developing, heart failure based, at least in part, on the presence of a mutation (e.g., c.67G>A single nucleotide variant) in both copies of a KCNJ11 gene present in the human). In some cases, a therapeutic agent used to treat heart failure can be an angiotensin-converting enzyme (ACE) inhibitor. In some cases, a therapeutic agent used to treat heart failure can be an angiotensin II receptor blocker. In some cases, a therapeutic agent used to treat heart failure can be a beta blocker. In some cases, a therapeutic agent used to treat heart failure can be a diuretic. In some cases, a therapeutic agent used to treat heart failure can be an aldosterone antagonist. In some cases, a therapeutic agent used to treat heart failure can be an angiotensin-neprilysin inhibitors. In some cases, a therapeutic agent used to treat heart failure can be a mineralocorticoid receptor antagonists. In some cases, a therapeutic agent used to treat heart failure can be a vasopressin receptor antagonists. Examples of therapeutic agents used to treat heart failure that can be administered to a mammal having, or at risk of developing, heart failure together with nucleic acid encoding a Kir6.2-E23 polypeptide (or Kir6.2-E23 polypeptides) include, without limitation, enalapril, Lisinopril, captopril, losartan, valsartan, carvedilol, metoprolol, bisoprolol, furosemide, spironolactone, eplerenone, inotropes, digoxin, hydralazine-isosorbide dinitrate, and ivabradine. In some cases, the one or more additional therapeutic agents can be administered together with nucleic acid encoding a Kir6.2-E23 polypeptide (or Kir6.2-E23 polypeptides; e.g., in the same composition). In some cases, the one or more additional therapeutic agents can be administered independent of the nucleic acid encoding a Kir6.2-E23 polypeptide (or Kir6.2-E23 polypeptides). When the one or more additional therapeutic agents are administered independent of the nucleic acid encoding a Kir6.2-E23 polypeptide (or Kir6.2-E23 polypeptides), the nucleic acid encoding a Kir6.2-E23 polypeptide (or Kir6.2-E23 polypeptides) can be administered first, and the one or more additional therapeutic agents administered second, or vice versa.

In some cases, the methods and materials described herein can include subjecting a mammal having, or at risk of developing, heart failure (e.g., a human having, or at risk of developing, heart failure based, at least in part, on the presence of a mutation (e.g., c.67G>A single nucleotide variant) in both copies of a KCNJ11 gene present in the human) to one or more (e.g., one, two, three, four, five or more) additional treatments (e.g., therapeutic interventions) that are effective to treat heart failure. Examples of additional treatments that can be used as described herein to treat heart failure include, without limitation, coronary bypass surgery, heart valve repair or replacement surgery (e.g., annuloplasty), implantation of an implantable cardioverter-defibrillators (ICDs), cardiac ablation, cardiac resynchronization therapy (CRT), intra-aortic balloon pump, implantation of ventricular assist devices (VADs), heart transplant, lifestyle changes (e.g., abstinence from smoking, fluid restriction, increased physical activity, adequate body mass index control), and/or dietary changes (e.g., reduced sodium consumption, maintaining healthy cholesterol, blood pressure, and glucose levels). In some cases, the one or more additional treatments that are effective to treat heart failure can be performed at the same time as the administration of the nucleic acid encoding a Kir6.2-E23 polypeptide (or Kir6.2-E23 polypeptides). In some cases, the one or more additional treatments that are effective to treat heart failure can be performed before and/or after the administration of the nucleic acid encoding a Kir6.2-E23 polypeptide (or Kir6.2-E23 polypeptides).

In some cases, the methods and materials described herein can be used to slow, delay, or reverse heart failure (e.g., slow, delay, or reverse the development of heart failure) in a mammal (e.g., a human having, or at risk of developing, heart failure based, at least in part, on the presence of a mutation (e.g., c.67G>A single nucleotide variant) in both copies of a KCNJ11 gene present in the human). For example, the methods and materials described herein can be used to reduce or eliminate one or more symptoms of heart failure. Examples of symptoms of heart failure that can be reduced or eliminated using the methods and materials described herein include, without limitation, shortness of breath (dyspnea; e.g., shortness of breath during exertion or shortness of breath during rest), fatigue, weakness, swelling (edema; e.g., swelling in the legs, ankles, and/or feet), rapid heartbeat, irregular heartbeat, reduced ability for activity, reduced ability to exercise, persistent cough or wheezing (e.g., persistent cough or wheezing with white or pink blood-tinged phlegm), increased need to urinate at night, lack of urine production, swelling of the abdomen (ascites), rapid weight gain (e.g., rapid weight gain from fluid retention), lack of appetite, nausea, difficulty concentrating, decreased alertness, sudden shortness of breath and coughing, severe shortness of breath and coughing, and trouble sleeping when lying flat (orthopnea). For example, the methods and materials described herein can be effective to reduce the severity of one or more symptoms of heart failure within a mammal (e.g., a human having, or at risk of developing, heart failure based, at least in part, on the presence of a mutation (e.g., c.67G>A single nucleotide variant) by, for example, 10, 20, 30, 40, 50, 60, 70, 80, 90, 95, or more percent.

In some cases, the methods and materials described herein can be used to delay the onset of one or more symptoms of heart failure in a mammal in a mammal (e.g., a human having, or at risk of developing, heart failure based, at least in part, on the presence of a mutation (e.g., c.67G>A single nucleotide variant) in both copies of a KCNJ11 gene present in the human). For example, the onset of one or more symptoms of heart failure in a mammal having a c.67G>A single nucleotide variant in both copies of a KCNJ11 gene present in the mammal can be delayed by from about 2 years to about 20 years (e.g., as compared to a mammal with a c.67G>A single nucleotide variant in both copies of a KCNJ11 gene that is not treated as described herein). In some cases, the onset of one or more symptoms of heart failure in a mammal having a c.67G>A single nucleotide variant in both copies of a KCNJ11 gene present in the mammal can be delayed by about 10 years (e.g., 10 heart failure free years) as compared to a mammal with a c.67G>A single nucleotide variant in both copies of a KCNJ11 gene that is not treated as described herein.

In some cases, the methods and materials described herein can be used to extend the life expectancy of a mammal in a mammal (e.g., a human having, or at risk of developing, heart failure based, at least in part, on the presence of a mutation (e.g., c.67G>A single nucleotide variant) in both copies of a KCNJ11 gene present in the human). For example, the life expectancy of a mammal having a c.67G>A single nucleotide variant in both copies of a KCNJ11 gene present in the mammal can be extended by from about 2 years to about 20 years or longer (e.g., as compared to the life expectancy of a mammal with a c.67G>A single nucleotide variant in both copies of a KCNJ11 gene that is not treated as described herein). For example, the life expectancy of a mammal having a c.67G>A single nucleotide variant in both copies of a KCNJ11 gene present in the mammal can be extended by about 10 years (e.g., 10 heart failure free years) as compared to the life expectancy of a mammal with a c.67G>A single nucleotide variant in both copies of a KCNJ11 gene that is not treated as described herein.

The invention will be further described in the following examples, which do not limit the scope of the invention described in the claims.

### EXAMPLES

### Example 1: KCNJ11 Genotype and Heart Failure Risk

In the heart failure epidemic, acquired risks do not fully account for disease susceptibility suggesting genetic modifiers.

This Example determined the impact of Kir6.2-E23K genotype on long-term risk for heart failure in a community-based outcome study. A community cohort (n=2031) was genotyped for the common KCNJ11-encoded Kir6.2-E23K variant of cardioprotective ATP-sensitive potassium channels. Impact was tested by heart failure surveillance over two decades, integrated into risk assessment, and validated in induced pluripotent stem cell-derived cardiomyocytes.

### Methods

### Participants

Residents 45 years of age or older were eligible. Participants provided written informed consent under an Institutional Review Board protocol. Random sampling achieved a fraction of 7% within each sex and age stratum. Of the 4203 contacted individuals, 2042 unrelated men and women were enrolled over a period of 45 months and completed the initial clinical examination with 2031 persons genotyped. Individuals with pre-existing heart failure (23 or 1.1%) were excluded from follow-up analysis.

### Genotyping

Genotyping targeted the KCNJ11-encoded c.67G>A variant (rs5219; Kir6.2 p.E23K). Genomic DNA was extracted from white blood cells (DNA isolation kit, Gentra Puregene) to amplify by the polymerase chain reaction a 407 bp fragment comprising the KCNJ11 variant using forward (5′-CCACGTCCGAGGGGTGC-3′ (SEQ ID NO:49)) and reverse (5′-AGGAGTGGATGCTGGTGACACA-3′ (SEQ ID NO:50)) primers. The amplicon was digested with the BanII restriction enzyme (New England Biolabs), and genotypes (reference allele homozygosity, EE; heterozygosity, EK; minor allele homozygosity, KK) assigned based on resultant fragment sizes resolved on 2% agarose gels, determined by presence (c.67G) or absence (c.67A) of a BanII recognition site. A second BanII site, unaltered by the variant, served as a control for complete digestion. Sanger sequencing validated representative restriction enzyme-derived genotypes.

### Evaluation

Surveillance included initial clinical examination, study-directed re-examination (4.0±0.3 years) and passive long-term follow-up. Linked electronic medical records across providers enabled reliable indexing of all hospital and clinic visits. Maximum follow-up was 20.6 years (median, 17.4; interquartile range, 13.3 to 18.3 years). Clinical examination and re-examination consisted of a self-administered questionnaire, anthropometric and blood pressure measurements, and transthoracic echocardiography (acquired by three sonographers and interpreted by a blinded, certified echocardiologist). Physician established clinical characteristics at study entry included history of diabetes, hypertension and coronary artery disease, along with heart failure diagnosis based on Framingham Heart Study criteria. Heart failure incidence (diagnosed in the clinic or hospital) was retrieved by a validated approach using codes 428 and 150 of the International Classification of Diseases (ICD-9 and ICD-10), supplemented by natural language processing algorithms. Each case was reviewed independently, and in duplicate, by three investigators masked to patients' characteristics and genotype. Inter-observer agreement was excellent (κ coefficient=0.91). Myocardial infarction cases were identified as ICD-9 410 and ICD-10 I21.9. Cardiovascular (ICD-9 codes 390 to 398, 402, and 404 to 429 and ICD-10 codes I00 to I09, I11, I13, and I20 to I51) and heart failure (ICD-9 428 and ICD-10 I50) associated deaths were obtained from County and nationwide civil sources.

### Induced Pluripotent Stem Cell-Derived Cardiomyocytes

Heart failure-free male and female donors, 23 to 51 years-old, provided tissue samples for derivation of induced pluripotent stem cells (Mayo Clinic Center for Regenerative Medicine Biotrust). Biopsied skin fibroblasts were reprogrammed using a Sendai virus-based approach, karyotyped, screened for pluripotency by quantitative polymerase chain reaction, and genotyped for Kir6.2-E23K. For each genotype (EE, EK or KK), three induced pluripotent stem cell clones verified for karyotype and pluripotency underwent cardiogenic differentiation. Real time metabolic profiles were evaluated with a Seahorse XF24 Extracellular Flux Analyzer (Agilent Technologies) in unbuffered media (25 mM glucose; 1 mM sodium pyruvate; 2 mM glutamax; lx nonessential amino acids; 1% FBS; pH 7.4). Calcium dynamics, assessed by confocal imaging (LSM 5 Live microcope, Carl Zeiss Microscopy GmbH) in Tyrode (in mM: 137 NaCl; 5.4 KCl; 1.8 CaCl₂); 1 MgCl₂; 5.5 glucose and 10 HEPES; pH 7.3) following cell loading with the Fluo-4AM calcium dye (Invitrogen), were analyzed with in-house coded Matlab software (MathWorks) algorithms. Apoptotic cell death was probed with the In Situ Cell Death Detection Kit, TMR red (Roche Diagnostics). Counterstaining using α-actinin antibody (1:200; Sigma A7811) documented sarcomeric structures. Whole-cell patch clamp electrophysiology was conducted in Tyrode with pipettes backfilled with (in mM) 140 KCl, 1 MgCl₂, 5 EGTA, 5 MgATP and 10 HEPES (pH 7.3). Action potentials were evoked in current-clamp using Bioquest software.

### Statistical Analysis

Continuous variables were provided as mean±SD and categorical variables as percentages. Categorical data were compared across groups using Pearson's chi-squared test. Comparison of continuous variables used ANOVA or Student's t-test. Time-to-event analysis testing association between genotypes and study outcomes was performed with the Kaplan Meier method, Log-rank test and Cox proportional hazard models. Modeling included adjustment for traditional risk factors and assessment of multiplicative interactions. Hazard ratios with 95% confidence intervals [CI] are reported. Population-attributable hazard was estimated from [100×Prevalence×(HR−1)]/[Prevalence x (HR−1)+1], with Prevalence denoting person-years for a given risk factor divided by the total person-years. Heart failure susceptibility was calculated based on one, two or three acquired risk factors considering genotype status. JMP 14.1 Statistical Discovery Software (SAS Institute) and SigmaPlot 11.0 (Systat Software) were used. Two-sided P values of less than 0.05 were considered to indicate statistical significance.

### Results

### Baseline Characteristics Across Kir6.2 Genotypes

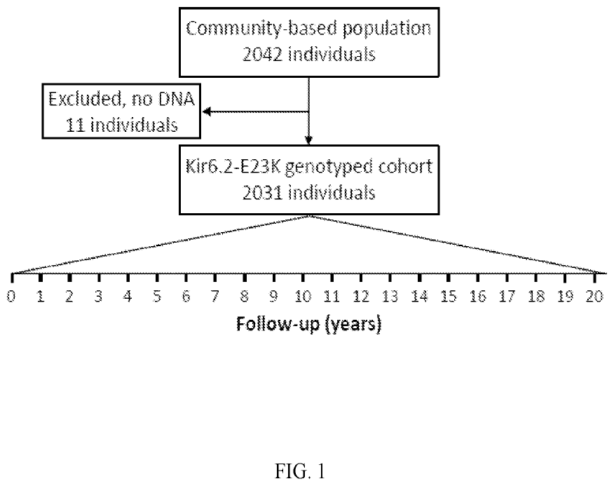

In a community-based cohort, 2042 individuals were randomly selected and 2031 successfully genotyped for Kir6.2-E23K, revealing 9% as KK homozygotes (FIG. 1 and Table 1). At study onset, no significant difference was observed in age, sex, body mass index, blood pressure or left ventricular ejection fraction across Kir6.2 genotypes (Table 1). Hypertension, diabetes and coronary artery disease prevalence were comparable (Table 1). Attrition over the 20-year follow-up was ≤0.6% (12 of 2031 participants).

TABLE 1

| | All | | EE | | EK | | KK | | |
|---|---|---|---|---|---|---|---|---|---|
| | (n = 2031) | | (n = 885) | | (n = 960) | | (n = 186) | P Value | |
| Men | 977 | (29.1%) | 434 | (49.0%) | 454 | (47.3%) | 89 (47.9%) | | 0.75 |
| Age, years | 62.2 ± 10.6 | | 62.3 ± 10.7 | | 62.0 ± 10.6 | | 62.2 ± 10.3 | | 0.34 |
| <55 - no. (%) | 598 | (29.4%) | 255 | (28.8%) | 295 | (30.7%) | 48 (25.8%) | | |
| <55-64 - no. (%) | 622 | (30.6%) | 271 | (30.6%) | 298 | (31.0%) | 53 (28.5%) | | |
| <65-74 - no. (%) | 513 | (25.3%) | 227 | (25.7%) | 225 | (23.5%) | 61 (32.8%) | {close oversize brace} | 0.28 |
| ≥72 - no. (%) | 298 | (14.7%) | 132 | (14.9%) | 142 | (14.8%) | 24 (12.9%) | | |
| Body mass Index, kg/m² | 28.6 ± 7.5 | | 28.7 ± 7.5 | | 28.6 ± 7.7 | | 28.4 ± 5.5 | | 0.92 |
| <25 - no. (%) | 511 | (25.2%) | 220 | (24.9%) | 236 | (24.6%) | 55 (29.6%) | | |
| <25-30 - no. (%) | 859 | (42.3%) | 378 | (42.7%) | 412 | (42.9%) | 69 (37.1%) | {close oversize brace} | 0.57 |
| ≥30 - no. (%) | 661 | (32.5%) | 287 | (32.4%) | 312 | (32.5%) | 62 (33.3%) | | |
| Systolic, mmHg | 133 ± 21 | | 133 ± 22 | | 132 ± 21 | | 133 ± 22 | | 0.55 |
| Diastolic, mmHg | 74 ± 10 | | 74 ± 11 | | 73 ± 10 | | 74 ± 11 | | 0.43 |
| Left ventricular ejection fraction, % | 63 ± 7 | | 63 ± 7 | | 63 ± 7 | | 62 ± 8 | | 0.40 |
| Hypertension -no. (%) | 766 | (37.7%) | 335 | (37.9%) | 355 | (37.0%) | 76 (40.9%) | | 0.61 |
| Diabetes -no, (%) | 149 | (7.3%) | 62 | (7.0%) | 67 | (7.0%) | 20 (10.8%) | | 0.21 |
| Coronary artery disease -no. (%) | 247 | (12.2%) | 110 | (12.4%) | 105 | (10.9%) | 32 (17.2%) | | 0.07 |

Plus-minus values are means are means ± SD

### Heart Failure Burden in KK Carriers

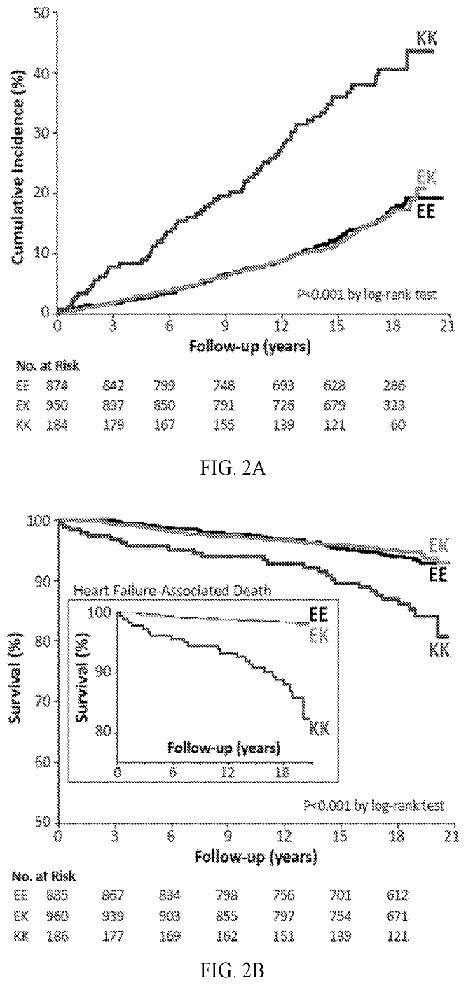

Surveillance of 2008 subjects (30369 person-years), who were heart failure-free at study onset, unmasked accelerated disease accumulation in KK carriers with a 20-year Kaplan-Meier heart failure incidence event rate (43.60%) exceeding by over two-fold that of EE (19.31%) or EK (20.82%) counterparts (log-rank P<0.001; FIG. 2A). At two decades, the KK genotype was present in 19% of subjects with heart failure vs. 7% without (P<0.001; Table 2). In fact, KK was overrepresented among genotypes (KK: 36%; EE: 15%, EK: 15%; P<0.001) in the 346 incident heart failure cases. Follow-up echocardiography supported the clinical diagnosis of heart failure with KK-heart failure cases associated with a higher 10 prevalence of preserved ejection fraction (Table 3). Notably, the KK genotype was linked to increased cardiovascular and heart failure-associated mortality rates (FIG. 2B, Table 4; log-rank P<0.001). Thus, KK carriers were more likely to develop heart failure and have poor outcome in the studied community.

TABLE 2
Genotype and Heart Failure Status.

| | | |
|---|---|---|
| no HF | HF | |
| (n = 1662) | (n = 346) | P Value |

KCNJ11 E23K Genotype

| | | | | | |
|---|---|---|---|---|---|
| EE - no. (%) | 739 | (44.5%) | 135 | (39.0%) | <0.001 |
| EK - no. (%) | 806 | (48.5%) | 144 | (41.6%) | <0.001 |
| KK - no. (%) | 117 | (7.0%) | 67 | (19.4%) | <0.001 |

Clinical Characteristics

| Age, years | 61.2 ± 10.3 | | 67.1 ± 10.4 | | <0.001 |
|---|---|---|---|---|---|
| Men - no. (%) | 785 | (47.2%) | 181 | (52.3%) | 0.68 |
| Hypertension - no. (%) | 592 | (35.6%) | 161 | (46.5%) | <0.001 |
| Diabetes - no. (%) | 95 | (5.7%) | 49 | (14.2%) | <0.001 |
| Coronary artery | 156 | (9.4%) | 77 | (22.3%) | <0.001 |
| disease - no. (%) | | | | | |

Comparison of subjects that developed versus those that did not develop heart failure (HF) over the 20 years follow-up. Plus - minus values are means ± SD.

TABLE 3
Echocardiographic Characteristics of Individuals
with Incident Heart Failure on Follow-up.
Echocardiographic Characteristics of Individuals
with Incident Heart Failure on Follow-up.

| | | | |
|---|---|---|---|
| All | EE/EK | KK | |
| (n = 337) | (n = 276) | (n = 61) | P Value |

Echocardlography

| Left ventricular | 55 ± 14 | | 54 ± 14 | | 60 ± 10 | | <0.001 |
|---|---|---|---|---|---|---|---|
| ejection fraction, % | | | | | | | |
| <40 - no. (%) | 52 | (15.4%) | 49 | (17.7%) | 3 | (4.9%) | <0.01 |
| ≥40 and <50 - no. (%) | 46 | (13.7%) | 35 | (12.7%) | 11 | (18.0%) | 0.02 |
| ≥50 - no. (%) | 239 | (70.9%) | 192 | (69.6%) | 47 | (77.1%) | 0.04 |
| Left ventricular end diastolic | 7.07 ± 7.11 | | 6.66 ± 6.47 | | 8.91 ± 9.36 | | 0.13 |
| dimension index, mm/m | | | | | | | |
| Left atrium volume | 42.37 ± 18.94 | | 43.18 ± 13.75 | | 39.29 ± 24.24 | | 0.44 |
| index, mL/m² | | | | | | | |
| E, m/s | 0.86 ± 0.31 | | 0.86 ± 0.32 | | 0.82 ± 0.28 | | 0.83 |
| E:e′ ratio | 14.98 ± 7.20 | | 15.12 ± 7.42 | | 14.31 ± 6.11 | | 0.42 |
| ≥10 - no. (%) | 188 | (76.4%) | 153 | (75.7%) | 35 | (79.6%) | 0.59 |

Parameters recorded upon or after heart failure diagnosis. E: early mitral inflow velocity: e′: mitral annular early diastolic velocity. Data are presented as number of individuals (%) unless otherwise noted. P values calculated when comparing individuals with EE/EK versus KK genotypes. Left ventricular ejection fraction could be measured in 337 participants (EE/EK: 276, KK: 61) Left ventricular end diastolic dimension and left atrial volume index could be measured in 243 participants (EE/EK: 199, KK: 44). E could be measured in 272 participants (EE/EK: 225, KK; 47). E/e′ could be measured in 246 participants (EE/EK: 202, KK: 44) Plus-minus values are means ± SD.

TABLE 4
Linkage of KK Genotype to Cardiovascular and Heart Failure Associated Mortality.
Linkage of KK Genotype to Cardiovascular and Heart Failure Associated Mortality.

| | Unadjusted | | | Adjusted for Age and Sex | | | Multivariate Model | | |
|---|---|---|---|---|---|---|---|---|---|
| | Hazard | (95% | P | Hazard | (95% | P | Hazard | (95% | P |
| | Ratio | CI) | Value | Ratio | CI) | Value | Ratio | CI) | Value |
| Cardiovascular Mortality | | | | | | | | | |
| Age, per 1 year increment | 1.14 | (1.12-1.16) | <0.001 | | | | 1.14 | (1.11-1.16) | <0.01 |
| Men | 1.57 | (1.09-2.25) | 0.01 | | | | 1.76 | (1.21-2.51) | 0.01 |
| Hypertension | 3.13 | (2.17-4.51) | <0.001 | 1.85 | (1.28-2.69) | <0.01 | 1.73 | (1.19-2.51) | <0.01 |
| Diabetes | 3.71 | (2.37-5.80) | <0.001 | 2.52 | (1.60-3.97) | <0.001 | 2.22 | (1.41-3.52) | <0.01 |
| Coronary artery disease | 5.68 | (3.90-8.29) | <0.001 | 2.28 | (1.53-3.40) | <0.001 | 1.93 | (1.30-2.89) | <0.01 |
| KK genotype | 2.68 | (1.72-4.16) | <0.001 | 2.69 | (1.73-4.18) | <0.001 | 2.70 | (1.73-4.20) | <0.001 |
| Heart Failure Associated | | | | | | | | | |
| Mortality | | | | | | | | | |
| Age, per 1 year increment | 1.15 | (1.12-1.19) | <0.001 | | | | 1.16 | (1.12-1.20) | <0.001 |
| Men | 1.42 | (0.80-2.51) | 0.20 | | | | 1.71 | (0.93-3.13) | 0.07 |
| Hypertension | 2.73 | (1.54-4.86) | <0.01 | 1.56 | (0.87-2.79) | 0.13 | 1.50 | (0.83-2.70) | 0.17 |
| Diabetes | 2.93 | (1.37-6.28) | <0.01 | 2.05 | (0.94-4.39) | 0.07 | 2.07 | (0.95-4.49) | 0.06 |
| Coronary artery disease | 5.70 | (3.14-10.34) | <0.001 | 2.22 | (1.18-4.17) | 0.01 | 1.72 | (0.91-3.26) | 0.09 |
| KK genotype | 8.69 | (4.92-15.37) | <0.001 | 8.64 | (4.90-15.25) | <0.001 | 8.60 | (4.84-15.28) | <0.001 |

Hazard ratios (95% confidence interval, CI) from unadjusted, adjusted for age/sex, and multivariate Cox proportional hazard models. Age is incorporated as a continuous variable. EE/EK genotype, denoting non-KK carrier status, is used as reference.

### KK Genotype Imposed Heart Failure Risk

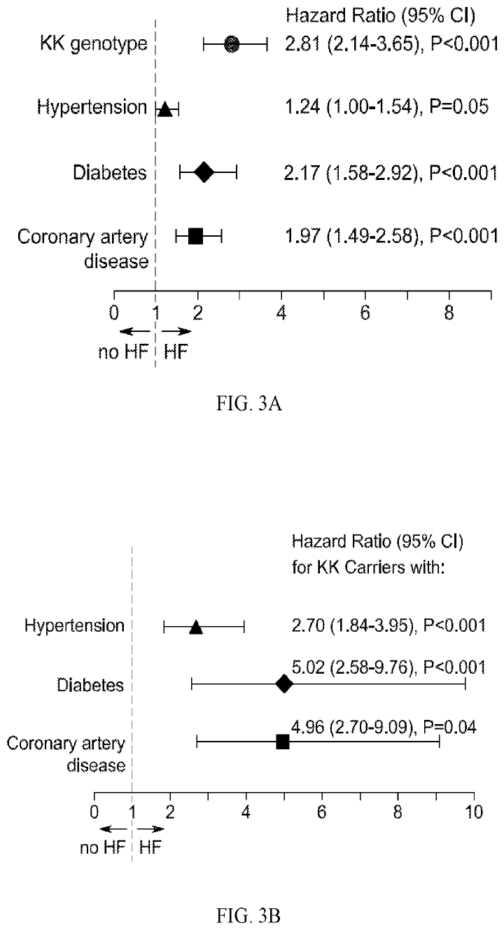

The KK genotype was predictive of heart failure (hazard ratio, 2.93; 95% CI, 2.17 to 3.91; P<0.00 and hazard ratio, 2.98; 95% CI, 2.21 to 3.96; P<0.00 vs. EL and EK, respectively), consistent after adjusting for age and sex (hazard ratio, 2.83; 95% CI, 2.10 to 3.78; P<0.001 and hazard ratio, 2.75; 95% CI, 2.04 to 3.65; P<0.001 vs. EE and EK, respectively; Table 5). In multivariable analysis, the predictive power of the KK genotype was independent of traditional risk factors including age, hypertension, diabetes and coronary artery disease (Table 1), exposing a heart failure hazard rate significantly and independently affected by KK compared to EE (hazard ratio, 2.77; 95% CI, 2.05 to 3.71; P<0.001) and EK (hazard ratio, 2.68; 95% CI, 1.99 to 3.58; P<0.001). KK-associated risk for heart failure adjusted for age and sex (hazard ratio vs. non-KK, 2.81; 95% CI, 2.14 to 3.65; P<0.001) exceeded that of hypertension (hazard ratio, 1.24; 95% CI, 1.00 to 1.54; P=0.05), diabetes (hazard ratio, 2.17; 95% CI, 1.58 to 2.92; P<0.001) or coronary artery disease (hazard ratio, 1.97; 95% CI, 1.49 to 2.58; P<0.001; FIG. 3A). KK attributable risk was further evidenced in subpopulations without hypertension (hazard ratio, 3.01; 95% CI, 2.08 to 4.25; P<0.001), diabetes (hazard ratio, 2.85; 95% CI, 2.12 to 3.76; P<0.001) or coronary artery disease (hazard ratio, 3.41; 95% CI, 2.53 to 4.53; P<0.001). Thus, the KK genotype is implicated in heart failure risk.

TABLE 5
KK Genotype Imposes Heart Failure Risk.
KK Genotype Imposes Heart Fallure Rlsk.

| | Unadjusted | | | Adjusted for Age and Sex | | | Multivariate Model | | |
|---|---|---|---|---|---|---|---|---|---|
| | Hazard | (95% | P | Hazard | (95% | P | Hazard | (95% | P |
| Variables | Ratio | CI) | Value | Ratio | CI) | Value | Ratio | CI) | Value |
| Age, years | | | | | | | | | |
| <55 | 1 | [Reference] | | | | | 1 | [Reference] | |
| 55-64 | 1.74 | (1.24-2.47) | <0.001 | | | | 1.49 | (1.06-2.13) | 0.02 |
| 65-74 | 3.52 | (2.55-4.91) | <0.001 | | | | 2.69 | (1.93-3.80) | <0.001 |
| ≥75 | 7.41 | (5.25-10.56) | <0.001 | | | | 5.87 | (4.09-8.51) | <0.001 |
| Men | 1.23 | (1.00-1.52) | 0.06 | | | | 1.23 | (0.99-1.53) | 0.06 |
| Hypertension | 1.76 | (1.42-2.17) | <0.001 | 1.47 | (1.17-1.86) | 0.01 | 1.20 | (0.96-1.50) | 0.10 |
| Diabetes | 2.86 | (2.09-3.83) | <0.001 | 2.19 | (1.59-2.95) | <0.001 | 1.93 | (1.40-2.61) | <0.001 |
| Coronary artery disease | 3.44 | (2.64-4.41) | <0.001 | 2.11 | (1.60-2.76) | <0.001 | 1.83 | (1.39-2.40) | <0.001 |
| KCNJ11 E23K genotype | | | | | | | | | |
| EE | 1 | [Reference] | | 1 | [Reference] | | 1 | [Reference] | |
| EK | 0.98 | (0.78-1.25) | 0.90 | 1.03 | (0.81-1.30) | 0.81 | 1.03 | (0.81-1.31) | 0.80 |
| KK | 2.93 | (2.17-3.91) | <0.001 | 2.83 | (2,10-3,78) | <0.001 | 2.77 | (2.05-3.71) | <0.001 |

Hazard ratios (95% confidence Interval, CI) from unadjusted, adjusted for age/sex, and multivariate Cox proportional hazard models. Age <55 years old and EE genotype are used as reference.

### Kir6.2 Genotyping and Risk Assessment

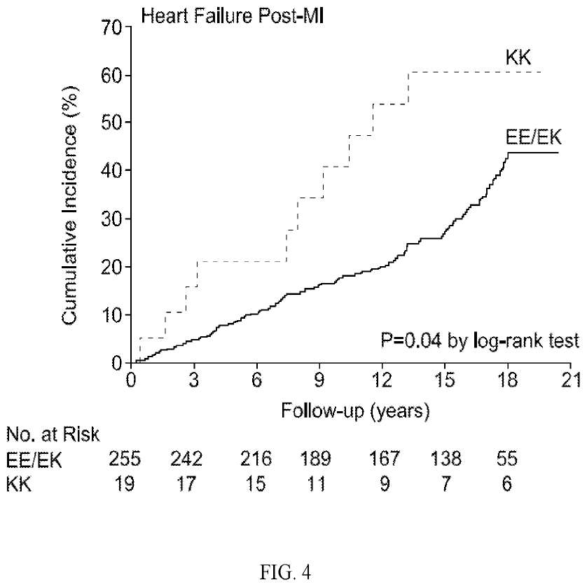

The KK genotype carried a high population-attributable hazard (12.16%; Table 6). KK status conferred incremental risk for heart failure in individuals with hypertension, diabetes or coronary artery disease (respective combined hazard ratio, 2.70; 95% CI, 1.84 to 3.95; P<0.001; 5.02; 95% CI, 2.58 to 9.76; P<0.001; 4.96; 95% CI, 2.70 to 9.09; P=0.04; FIG. 3B). Among subjects who had myocardial infarction, KK vs. non-KK carriers developed a greater heart failure event rate (60.53% and 43.75%, respectively, log-rank P=0.04; FIG. 4) and an increased risk for heart failure after adjusting for traditional risk factors (hazard ratio 2.10, 95% CI, 1.06 to 4.15; P=0.03; Table 7). KK genotype incorporated into heart failure susceptibility evaluation refined predicted risk (cumulative hypertension-diabetes-coronary artery disease hazard ratio, 4.47; 95% CI, 2.45 to 7.52 vs. 7.64; 95% CI, 1.88 to 31.05 in KK carriers; Table 8). Taken together, KK homozygosity accounted for an eighth of all heart failure cases in the study population, with Kir6.2 genotyping adding value to personalized risk assessment.

TABLE 6
Population Attributable Hazard for Developing Heart Failure.

| | | | |
|---|---|---|---|
| | | | Population- |
| Person- | Prevalence | Hazard Ratio | Attributable |
| Years | (%) | (95% CI) | Hazard (%) |

Risk Factors

| | | | | |
|---|---|---|---|---|
| Hypertension | 10358.4 | 34.1 | 1.20 (0.96-1.50) | 6.39 |
| Diabetes | 1739.1 | 5.7 | 1.93 (1.40-2.61) | 5.06 |
| Coronary | 2554.1 | 8.4 | 1.83 (1.39-2.40) | 6.52 |
| artery disease | | | | |
| KK genotype | 2374.4 | 7.8 | 2.77 (2.05-3.71) | 12.16 |

Prevalence denotes person-years for a given risk factor divided by the total person-years. Hazard ratios (HR; 95% confidence interval, CI) for heart failure adjusted for age, sex, hypertension, diabetes and coronary artery disease.
Population-attributable hazard (%) defined as [100 × Prevalence × (HR − 1)]/[Prevalence × (HR − 1) + 1].

TABLE 7
Heart Failure Risk Following Myocardial Infarction in KK vs. Non-KK Carriers.
Heart Failure Risk Following Myocardial Infarction in KK vs. Non-KK Carriers.

| | Unadjusted | | | Adjusted for Age and Sex | | | Multivariate Model | | |
|---|---|---|---|---|---|---|---|---|---|
| | Hazard | (95% | P | Hazard | (95% | P | Hazard | (95% | P |
| Characteristics | Ratio | CI) | Value | Ratio | CI) | Value | Ratio | CI) | Value |
| Age, per 1 year increment | 1.07 | (1.04-1.09) | <0.01 | | | | 1.07 | (1.04-1.09) | <0.01 |
| Men | 1.29 | (0.85-1.94) | 0.20 | | | | 1.03 | (0.67-1.57) | 0.80 |
| Hypertension | 1.19 | (0.80-1.78) | 0.40 | 1.03 | (0.69-1.56) | 0.80 | 1.03 | (0.68-0.55) | 0.80 |
| Diabetes | 1.23 | (0.71-2.13) | 0.40 | 1.23 | (0.71-2.14) | 0.40 | 1.10 | (0.62-1.94) | 0.70 |
| KK genotype | 1.97 | (1.03-3.81) | 0.04 | 2.16 | (1.12-4.18) | 0.02 | 2.10 | (1.06-4.15) | 0.03 |

Hazard ratios (95% confidence Interval, CI) from unadjusted, adjusted for age/sex, and multivarlate Cox proportional hazard models following acute myocardial infarction, Age is incorporated as a continuous variable. EE/EK genotype, denoting non-KK carrler status, Is used as reference.

TABLE 8
Heart Failure Susceptibility Evaluation.

| | |
|---|---|
| Hazard Ratio | |
| (95% CI) | P Value |

Clinical Predictors
Acquired risk factor(s)

| | | |
|---|---|---|
| One | 1.46 (1.14-1.86) | <0.01 |
| Two | 1.92 (1.36-6.78) | <0.001 |
| Three | 4.47 (2.45-7.52) | <0.001 |

Clinical Predictors Plus Genotype Status
Acquired risk factor(s) in non-KK individuals

| | | |
|---|---|---|
| One | 1.31 (1.01-1.69) | <0.01 |
| Two | 1.74 (1.23-2.49) | <0.001 |
| Three | 4.18 (2.30-7.61) | <0.001 |

Acquired risk factor(s) in KK individuals

| | | |
|---|---|---|
| One | 2.95 (1.93-4.55) | <0.001 |
| Two | 3.81 (1.85-7.84) | <0.001 |
| Three | 7.64 (1.88-31.05) | <0.01 |

Age/sex adjusted hazard ratios (95% confidence interval, CI) calculated based on one, two or three acquired risk factors, namely hypertension, diabetes and/or coronary artery disease, not accounting for genotype status (top rows) and after incorporating genotype status (non-KK individuals, middle row; KK individuals, lower rows) in heart failure susceptibility assessment. Not accounting for genotype status, individuals with no acquired risk factors are used as reference.

### KK Genotype and Myocellular Distress

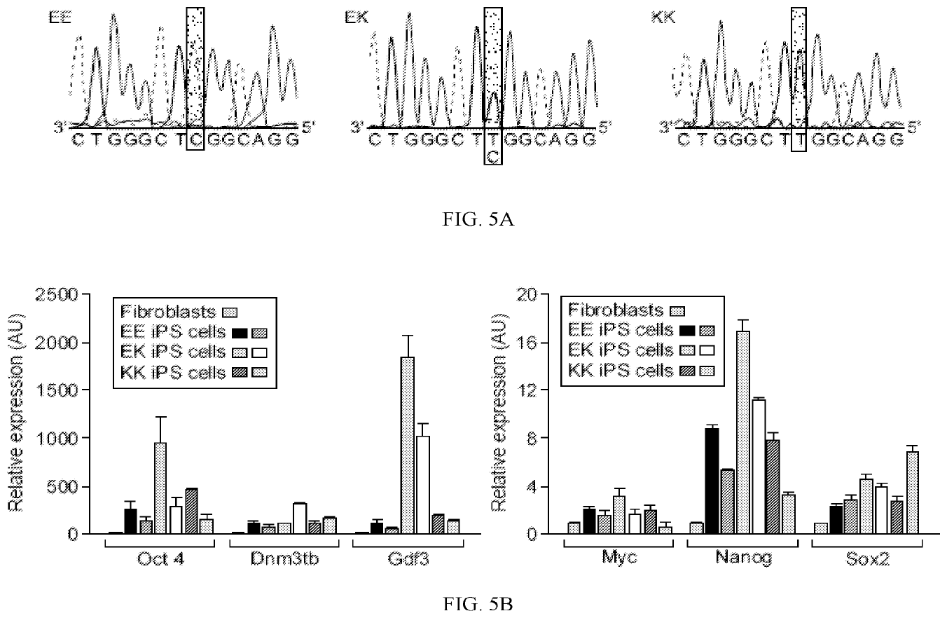

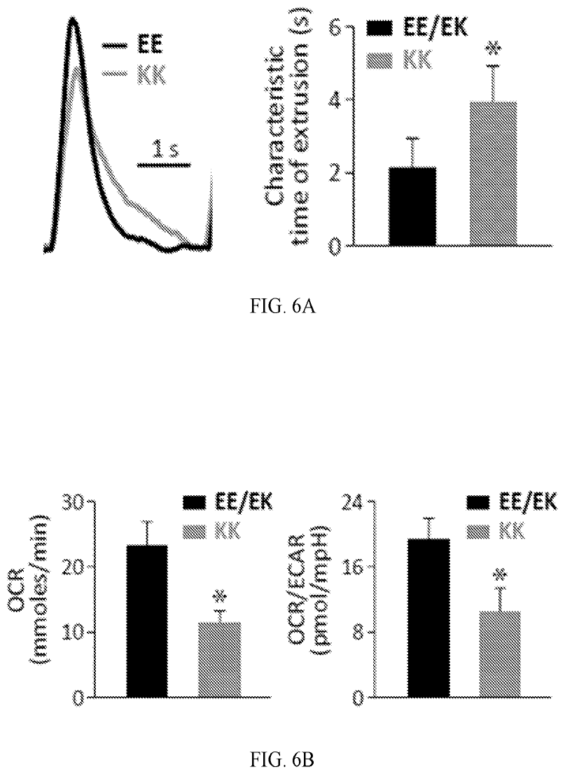

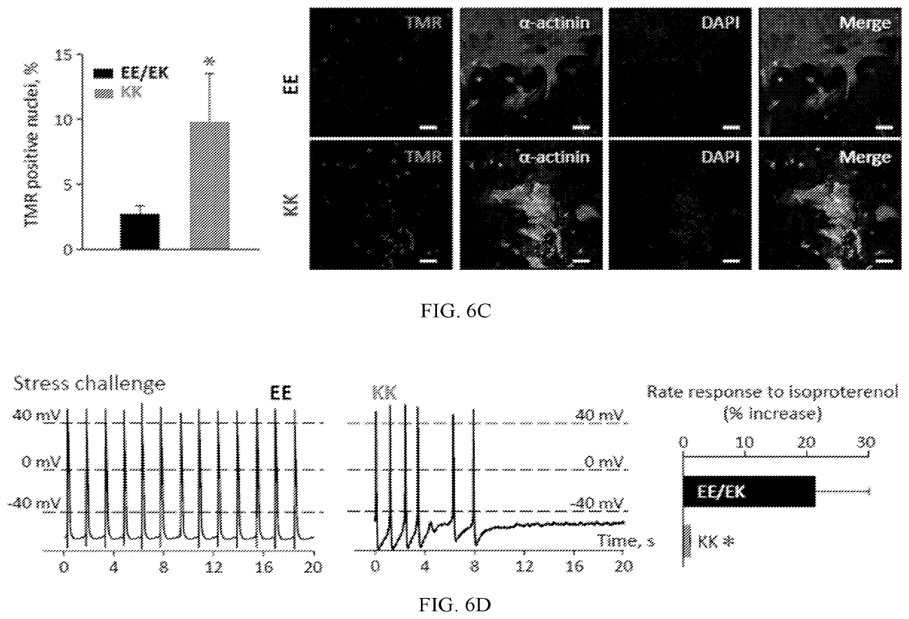

To independently test genotype impact, free of confounding variables, induced pluripotent stem cells harboring EE, EK or KK were engineered from heart failure-free donors (FIG. 5) and differentiated into functional cardiomyocytes. Derived cardiac cells generated contractile and electrical activity. Yet, compared to the EE/EK genotype, KK cells displayed evidence of calcium mishandling with variable rhythmicity and calcium transients characterized by a prolonged diastolic extrusion phase (2.16±0.76 s vs. 3.93±0.98 s, p=0.01; FIG. 6A). KK cardiomyocytes exhibited altered energetic use (FIG. 6B), and more prevalent apoptosis (9.73±3.82% in KK vs. 2.77±0.58% in EE/EK cells, p=0.01; FIG. 6C). Stress intolerant KK cardiomyocytes were unable to sustain adrenergic challenge, in contrast to EE/EK counterparts (FIG. 6D). Thus, a cell-autonomous model system validated that KK homozygosity confers a vulnerable phenotype with characteristics of a cardiomyopathic predilection.

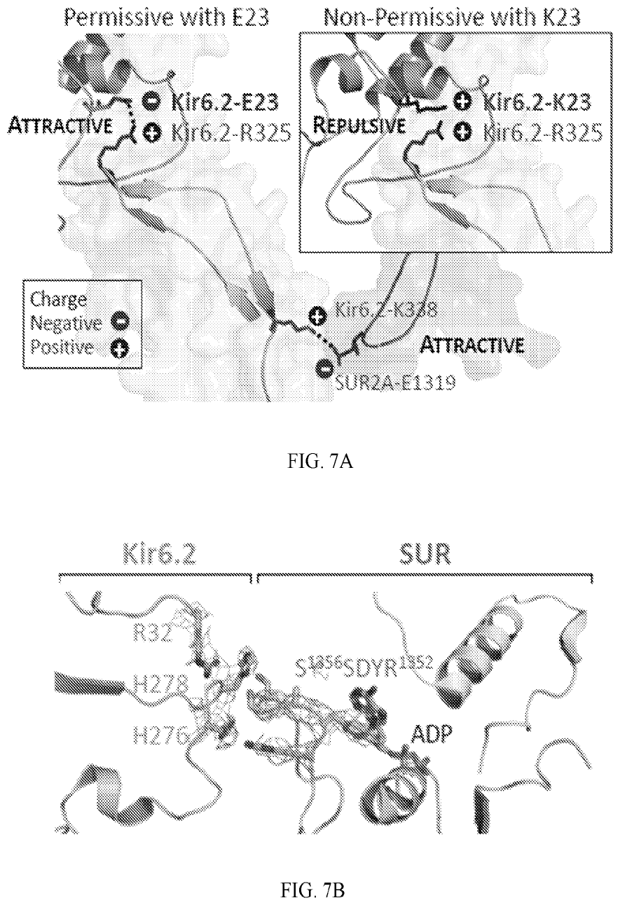

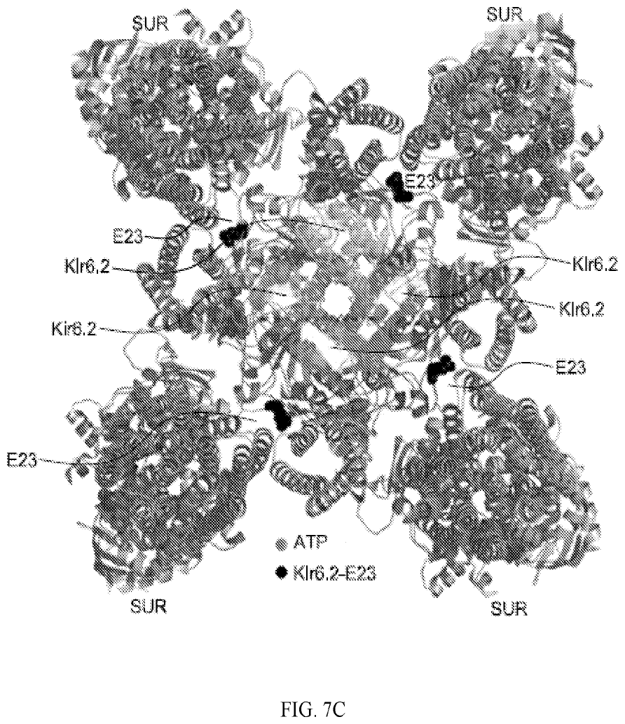

Taken together, these results demonstrate that the presence of a KK genotype can be used to identify a mammal (e.g., a human) as being likely to experience heart failure. For example, the positively charged K23 variant can compromise Kir6.2-Kir6.2 communication mediated by interaction between the negatively charged E23 and positively charged R325 residues (FIG. 7A). Further, insertion of K23 can impede the downstream R325/K338 Kir6.2 locale, precluding proper coupling of Kir6.2 with SUR2A via E1319 (FIG. 7A). Functionally, the Kir6.2-K23 K_{ATP} channel variant can display abnormal gating in response to metabolic regulation, reflective of improper communication with SUR-bound adenine nucleotides (FIG. 7B). Multimeric K_{ATP} channel stoichiometry, comprising four Kir6.2 plus four SUR2A subunits (FIG. 7C), can predict retained gating properties in >90% of channels in EK heterozygotesotes.

### Example 2: Homozygous KK Status Aggravates Heart Failure Outcomes

Homozygosity for the KCNJ11-encoded Kir6.2-E23K (KK) can be associated with increased risk for developing heart failure (see, e.g., Example 1). This Example evaluates the impact of the KK genotype on clinical outcomes in patients with heart failure.

### Methods

Consecutive patients with heart failure that attended cardiovascular health clinics were enrolled after providing inform consent. Participants underwent echocardiography, and blood draw for neurohumoral assays and genotyping for the KCNJ11 c.67G>A encoding Kir6.2 p.E23K. Record linkage enabled prospective surveillance for cardiovascular and heart failure related readmission and mortality.

### Results

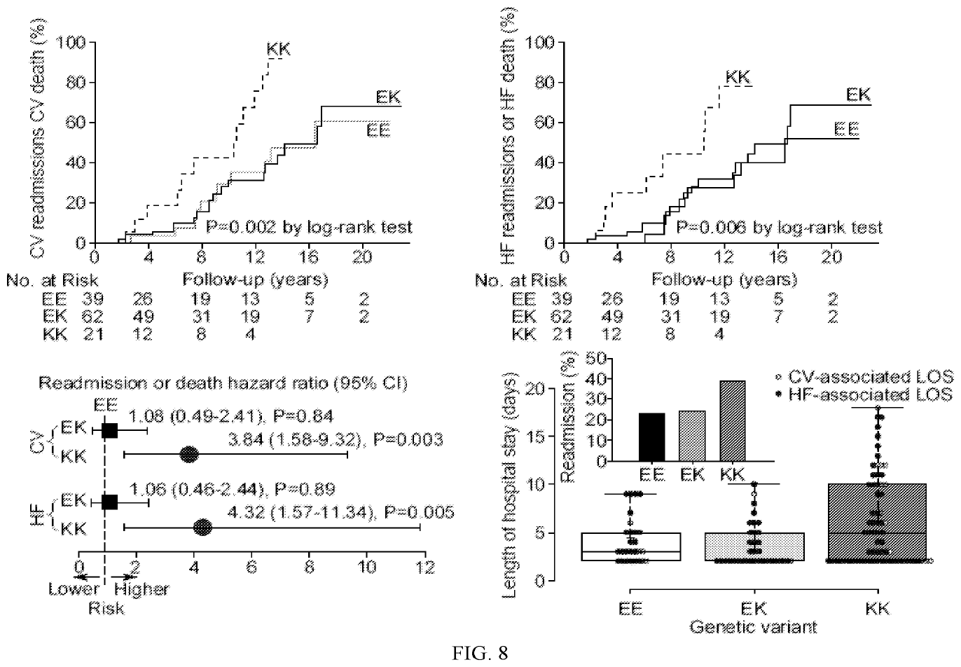

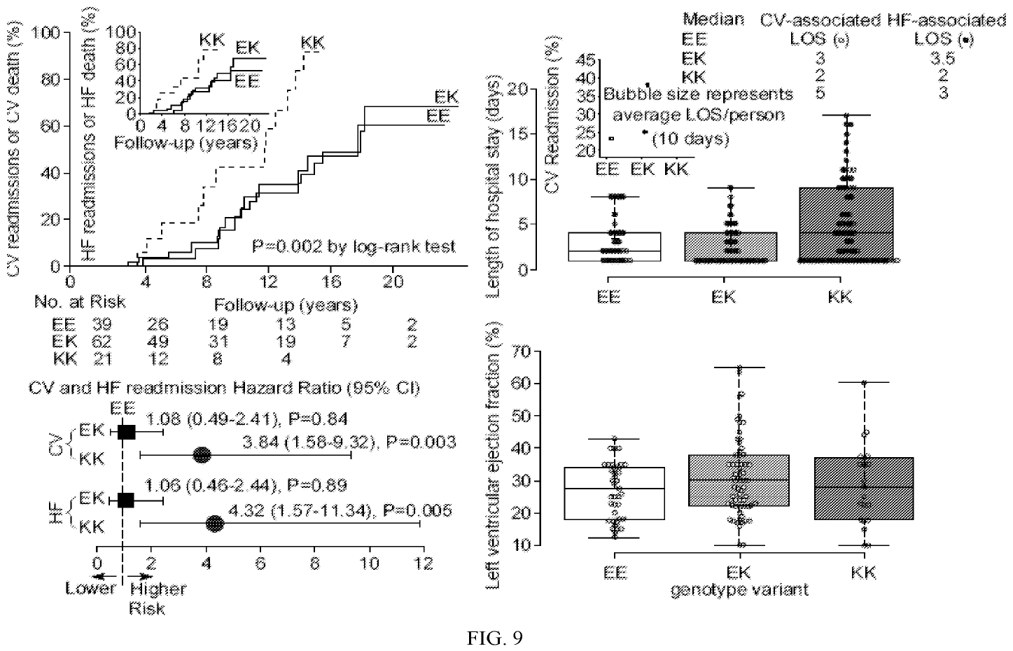

The 122 individuals [81 (66%) men, mean age±SD 55.7±12.4 years] were diagnosed with reduced ejection fraction (EF 29.3±11.4%) heart failure. Genotyping revealed 21 (17%) KK, 39 (32%) EE, and 62 (51%) EK subjects. KK status was associated with accelerated cardiovascular [Hazard ratio (HR) 3.84 95% CI 1.58-9.32), P=0.003] and heart failure (HR 4.32, 95% CI 1.57-11.34, P=0.005) related readmissions (FIG. 8). Likewise, KK homozygotes experienced a doubling of readmissions compared to EE/EK. When admitted, KK individuals required a prolonged length of stay for cardiovascular (KK 6.0±4.7, EK 3.6±2.2 and EE 4.2±2.4; p=0.003) and heart failure related (KK 5.6±4.5, 3.4±2.0, and EE 4.4±2.5; p=0.02) admissions (FIG. 9).

These results demonstrate that homozygous K_{ATP} Kir6.2 channel pore KK status can be used to identify a mammal (e.g., a human) at risk for poor heart failure outcome (e.g., as documented by increased and prolonged hospital readmissions and earlier death in a diseased cohort).

### Example 3: E23K Genotype Reclassifies Individuals at Risk for Heart Failure Offering Theranostic Value

Current guidelines recommend traditional modifiable factors and natriuretic peptide biomarkers in heart failure screening. Deduced profiles reflect hearts-at-risk, without considering intrinsic cardiac susceptibility. This Example evaluates the utility of the non-modifiable Kir6.2-K23 genetic variant on heart failure risk stratification.

### Methods

In a random Olmsted county community, residents aged 45 years or older underwent genotyping for KCNJ11 c.67G>A encoding Kir6.2 p.E23K, along with clinical and natriuretic peptide (NT-proBNP) assessment. Impact of KK carrier status on heart failure risk was assessed in high vs low risk individuals (i.e., those with vs without hypertension, diabetes, coronary artery disease and/or elevated body mass index ≥30 Kg/m²), as well as in those with normal vs clinically elevated NT-proBNP (>125 pg/mL). Kaplan-Meier incident heart failure event rates, Cox proportional hazard models, and net reclassification indexes were computed.

### Results

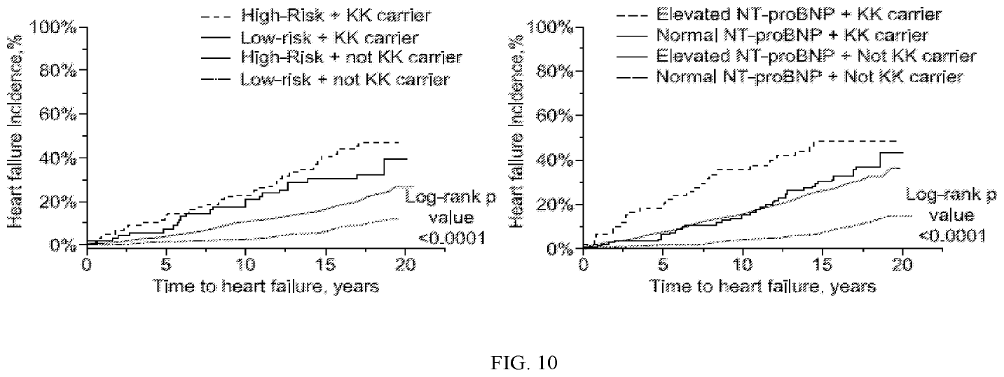

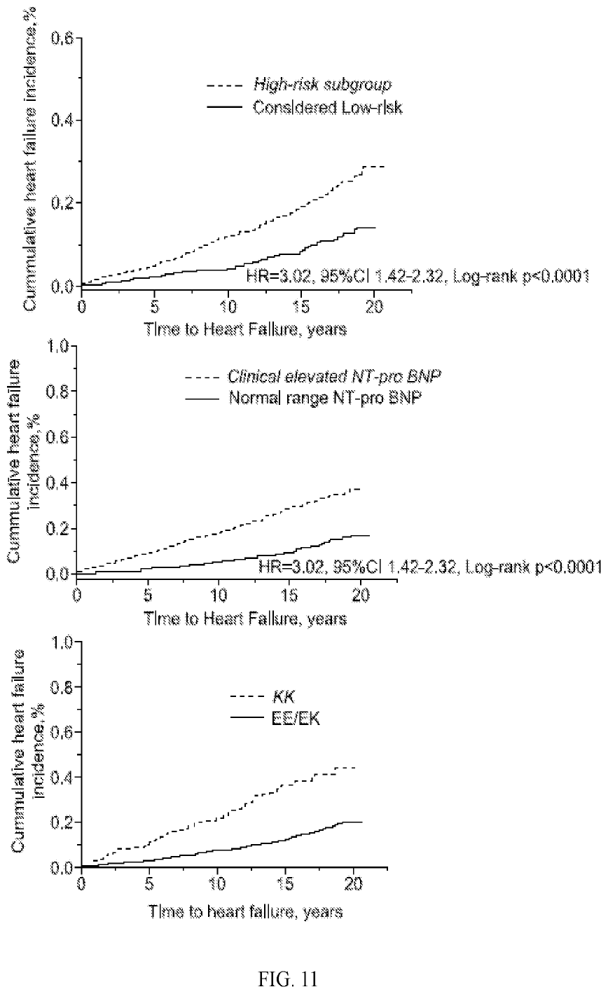

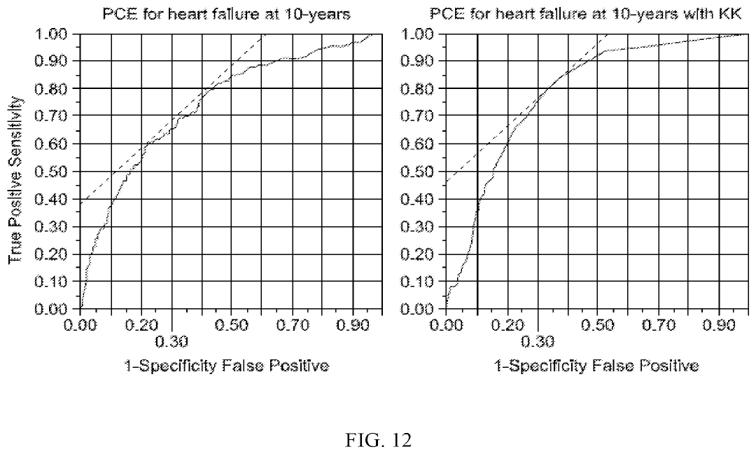

Surveillance included 1961 individuals (30369 person-years), with 52% women, mean age±SD 62.1±10.5 years, 1142 (58%) considered high-risk based on traditional risk factors, and 578 (29%) with elevated NT-proBNP (median 68.1 pg/mL, IQR 27.5-142.8). Over a two-decade follow-up (median 17.4 years, IQR 13.3-18.3), 337 (17%) developed heart failure. Of those, 242 (72%) were considered high-risk [Hazard ratio (HR) 1.77, 95% CI 1.39, 2.25], and 154 (45%) had elevated NT-proBNP (HR 1.80, 95% CI 1.41, 2.31). In the whole cohort, KK homozygosity present in 181 (9%) individuals was associated with tripled heart failure risk (HR 2.80, 95% CI 2.13, 3.66). Notably, for those traditionally considered at low risk, KK status unmasked heart failure susceptibility (HR 3.64 95% CI 2.26-5.87), and furthermore aggravated risk for high-risk individuals (HR 2.44 95% CI 1.76-3.39). Likewise, KK revealed susceptible individuals despite normal NT-proBNP (HR 2.00 95% CI 1.33-3.03), and amplified risk for those with pre-elevated NT-proBNP (HR 4.00 95% CI 2.56-6.25). Informing risk prediction, KK status incorporated in the pooled cohort equations (AUC 0.74 without vs 0.79 with KK, p<0.0001) reclassified 8.2% of the population vulnerable to heart failure. See, for example, FIGS. 10-14.

TABLE 9-A
Comparison of baseline characteristics between
the population and High risk subgroup.

| | Total | | High Risk | | |
|---|---|---|---|---|---|
| | Population | | sub-group | | |
| | (n = 1961) | | (n = 1142) | | p value |
| Age, years | 64.15 ± 10.15 | | 64.16 ± 10.68 | | 0.0876 |
| Sex (male)^{†} | 944 | (48.14) | 559 | (48.95%) | 0.8762 |
| Race/ethnicity (white) | 1909 | (97.65) | 1109 | (97.62%) | |
| BMI (kg/m²) | 28.64 ± 7.53 | | 30.83 ± 8.88 | | 0.7569 |
| Systolic blood pressure | 132.81 ± 21.49 | | 139.01 ± 71.70 | | 0.9387 |
| Diastolic blood pressure | 73.62 ± 10.27 | | 75.03 ± 10.61 | | 0.9942 |
| Heart rate | 66.35 ± 11.45 | | 67.12 ± 12.08 | | 0.1199 |
| Hypertension | 734 | (37.43) | 734 | (64.27%) | — |
| Diabetes mellitus | 141 | (7.19) | 141 | (12.35%) | — |
| Coronary Artery Disease | 230 | (11.73) | 230 | (20.14%) | — |

Echocardiography

| LVEF, mean (SD), % | 62.93 ± 6.95 | | 62.54 ± 7.85 | | 0.3631 |
|---|---|---|---|---|---|
| <40 | 26 | (1.33) | 24 | (2.12%) | |
| >/40-50 | 51 | (2.61) | 41 | (3.62%) | 0.2944 |
| >/50 | 1874 | (96.05) | 1068 | (94.26%) | |
| LVEDD/height, | 4.91 ± 0.52 | | 4.97 ± 0.54 | | 0.0734 |
| mean (SD), mm/m | | | | | |
| LAVI, mean (SD) | 24.57 ± 10.22 | | 26.15 ± 12.17 | | 0.8817 |
| E, m/s | 0.67 ± 0.16 | | 0.68 ± 0.18 | | 0.1882 |
| e′, m/s | 0.08 ± 0.03 | | 0.08 ± 0.03 | | 0.8034 |
| E:e′ ratio | 8.64 ± 3.09 | | 9.24 ± 3.29 | | 0.4826 |
| ≥10 | 470 | (29.21) | 342 | (36.97%) | 0.2519 |

Diastolic function

| | | | | | |
|---|---|---|---|---|---|
| Normal | 1240 | (70.57) | 600 | (60.46%) | |
| Mild | 349 | (19.86) | 257 | (25.91%) | 0.1014 |
| Moderate/Severe | 168 | (9.56) | 135 | (13.61%) | |

TABLE 9-B
Hazard Ratios for heart failure at 20-years.

| | Unadjusted | | | Age and Sex Adjusted | | | Multivariate Model | | |
|---|---|---|---|---|---|---|---|---|---|
| | Hazard | (95% | p- | Hazard | (95% | p- | Hazard | (95% | p- |
| | Ratio | CI) | value | Ratio | CI) | value | Ratio | CI) | value |
| Age, per 1 year Increase | 1.08 | 1.06-1.09 | <0.0001 | 1.06 | 1.05-1.08 | <0.0001 | 1.06 | 1.05-1.07 | <0.001 |
| Men | 1.25 | 1.01-1.54 | 0.0421 | 1.56 | 1.26-1.94 | <0.0001 | 1.36 | 1.08-1.70 | 0.0083 |
| Hypertension | 1.78 | 1.44-2.2 | <0.0001 | 1.25 | 1.01-1.56 | 0.0445 | 1.06 | 0.84-1.32 | 0.6338 |
| Diabetes | 2.89 | 2.14-3.92 | <0.0001 | 2.17 | 1.6-2.95 | <0.0001 | 1.35 | 0.74-2.44 | <0.001 |
| Coronary artery disease | 3.36 | 2.82-4.67 | <0.0001 | 2.06 | 1.57-2.69 | <0.0001 | 1.68 | 1.27-2.22 | <0.001 |
| BMI, per increase in 1 kg/m2 | 1.00 | 0.99-1.01 | 0.3489 | 1.01 | 1-1.02 | 0.0189 | 1.01 | 1.00-1.02 | 0.0375 |
| High Risk sub-group | 3.02 | 2.44-3.74 | <0.0001 | 1.96 | 1.54-2.5 | <0.0001 | 1.81 | 1.42-2.32 | <0.001 |

TABLE 10-A
Comparison of baseline characteristics between participants
with normal and clinical elevated NT-pro BNP.

| | Normal | | Elevated | | |
|---|---|---|---|---|---|
| | NT-ProBNP | | NT-ProBNP | | p value |
| Age, years | 58.90 ± 8.91 | | 69.94 ± 10.02 | | <0.0001 |
| Sex (male)^{†} | 648 | (46.85%) | 369 | (63.84%) | <0.0001 |
| Race/ethnicity (white) | 1346 | (97.47%) | 563 | (98.08%) | 0.4444 |
| BMI (kg/m²) | 28.94 ± 8.11 | | 27.92 ± 5.87 | | 0.0001 |
| Systolic blood pressure | 129.34 ± 18.41 | | 141.13 ± 25.67 | | <0.0001 |
| Diastolic blood pressure | 73.83 ± 9.88 | | 73.11 ± 11.15 | | 0.0415 |
| Heart rate | 66.39 ± 11.13 | | 66.24 ± 12.17 | | 0.4489 |
| Hypertension | 422 | (30.51%) | 312 | (53.98%) | 0.0001 |
| Diabetes mellitus | 79 | (5.71%) | 62 | (10.3%) | 0.0001 |
| Coronary Artery Disease | 92 | (6.65%) | 138 | (23.88%) | 0.0001 |

Echocardiography

| LVEF, mean (SD), % | 63.46 ± 5.47 | | 61.65 ± 9.49 | | 0.0001 |
|---|---|---|---|---|---|
| <40 | 1 | (0.07%) | 25 | (4.36%) | |
| >/40-50 | 16 | (1.16%) | 35 | (6.11%) | <0.0001 |
| >/50 | 1361 | (98.77%) | 513 | (84.53%) | |
| LVEDD/height, | 4.91 ± 0.46 | | 4.93 ± 0.63 | | 0.0765 |
| mean (SD), mm/m | | | | | |
| LAVI, mean (SD) | 22.71 ± 5.95 | | 29.15 ± 15.66 | | 0.0001 |
| E, m/s | 0.66 ± 0.14 | | 0.70 ± 0.21 | | 0.0001 |
| e′, m/s | 0.09 ± 0.04 | | 0.08 ± 0.03 | | 0.0001 |
| E:e′ ratio | 8.22 ± 2.69 | | 9.73 ± 3.71 | | 0.0001 |
| ≥10 | 282 | (24.33%) | 188 | (41.78%) | |

Diastolic function

| | | | | | |
|---|---|---|---|---|---|
| Normal | 1017 | (79.21%) | 223 | (47.15%) | |
| Mild | 208 | (16.20%) | 141 | (29.81%) | 0.0001 |
| Moderate/Severe | 59 | (4.60%) | 109 | (2.04%) | |

TABLE 10-B
Hazard Ratios for heart failure.

| | Unadjusted | | | Age and Sex Adjusted | | | Multivariate Model | | |
|---|---|---|---|---|---|---|---|---|---|
| | Hazard | (95% | p- | Hazard | (95% | p- | Hazard | (95% | p- |
| | Ratio | CI) | value | Ratio | CI) | value | Ratio | CI) | value |
| Age, per 1 year increase | 1.08 | 1.06-1.09 | <0.0001 | 1.06 | 1.05-1.08 | <0.0001 | 1.06 | 1.05-1.07 | <0.001 |
| Men | 1.25 | 1.01-1.54 | 0.0421 | 1.56 | 1.26-1.94 | <0.0001 | 1.36 | 1.08-1.70 | 0.0083 |
| Hypertension | 1.78 | 1.44-2.2 | <0.0001 | 1.25 | 1.01-1.56 | 0.0445 | 1.06 | 0.84-1.32 | 0.6338 |
| Diabetes | 2.89 | 2.14-3.92 | <0.0001 | 2.17 | 1.6-2.95 | <0.0001 | 1.35 | 0.74-2.44 | <0.001 |
| Coronary artery disease | 3.36 | 2.82-4.67 | <0.0001 | 2.06 | 1.57-2.69 | <0.0001 | 1.68 | 1.27-2.22 | <0.001 |
| BMI, per Increase In 1 kg/m2 | 1.00 | 0.99-1.01 | 0.3489 | 1.01 | 1-1.02 | 0.0189 | 1.01 | 1.00-1.02 | 0.0375 |
| Elevated NT-ProBNP | 3.02 | 2.44-3.74 | <0.0001 | 1.96 | 1.54-2.5 | <0.0001 | 1.81 | 1.42-2.32 | <0.001 |

TABLE 11-A
Comparison of baseline characteristics
in KK and non-KK individuals.

| | KK | | EK/EE | | p value |
|---|---|---|---|---|---|
| Age, years | 63.35 ± 10.31 | | 62.03 ± 10.55 | | 0.0876 |
| Sex (male)^{†} | 95 | (52.49%) | 922 | (51.80%) | 0.8598 |
| Race/ethnicity (white) | 173 | (95.58%) | 1736 | (97.86%) | 0.0028 |
| BMI (kg/m²) | 28.40 ± 5.48 | | 28.67 ± 7.71 | | 0.7569 |
| Systolic blood pressure | 132.92 ± 21.74 | | 132.80 ± 21.47 | | 0.9387 |
| Diastolic blood pressure | 73.48 ± 10.96 | | 73.63 ± 10.22 | | 0.9942 |
| Heart rate | 67.43 ± 11.38 | | 66.24 ± 11.45 | | 0.1199 |
| Hypertension | 72 | (39.78%) | 662 | (37.19%) | 0.4931 |
| Diabetes mellitus | 18 | (9.94%) | 123 | (6.91%) | 0.1321 |
| Coronary Artery Disease | 29 | (16.02%) | 201 | (11.29%) | 0.0595 |

Echocardiography

| LVEF, mean (SD), % | 62.25 ± 8.12 | | 63 ± 6.81 | | 0.3631 |
|---|---|---|---|---|---|
| <40 | 4 | (2.21%) | 22 | (1.24%) | |
| >/40-50 | 7 | (0.87%) | 44 | (2.49%) | 0.2944 |
| >/50 | 170 | (93.92%) | 1704 | (96.27%) | |
| LVEDD/height, | 4.99 ± 0.59 | | 4.90 ± 0.50 | | 0.0734 |
| mean (SD), mm/m | | | | | |
| LAVI, mean (SD) | 24.36 ± 8.03 | | 24.60 ± 10.42 | | 0.8817 |
| E, m/s | 0.68 ± 0.16 | | 0.67 ± 0.16 | | 0.8034 |
| e′, m/s | 0.08 ± 0.02 | | 0.08 ± 0.04 | | 0.4826 |
| E:e′ ratio | 8.94 ± 3.29 | | 8.61 ± 3.06 | | 0.1882 |
| ≥10 | 470 | (29.21%) | 342 | (36.97%) | 0.2253 |

Diastolic function

| | | | | | |
|---|---|---|---|---|---|
| Normal | 108 | (67.08%) | 1132 | (70.93%) | |
| Mild | 30 | (18.63%) | 319 | (19.99%) | 0.1014 |
| Moderate/Severe | 23 | (14.29%) | 145 | (9.09%) | |

TABLE 11-B
KK genotype imposes heart failure risk.

| | Unadjusted | | | Age and Sex Adjusted | | | Multivariate Model | | |
|---|---|---|---|---|---|---|---|---|---|
| | Hazard | (95% | p- | Hazard | (95% | p- | Hazard | (95% | p- |
| | Ratio | CI) | value | Ratio | CI) | value | Ratio | CI) | value |
| Age. per 1 year increase | 1.08 | 1.06-1.09 | <0.0001 | 1.06 | 1.05-1,08 | <0.0001 | 1.06 | 1.05-1.07 | <0.001 |
| Men | 1.25 | 1.01-1.54 | 0.0421 | 1.56 | 1.26-1.94 | <0.0001 | 1.36 | 1.08-1.70 | 0.0083 |
| Hypertension | 1.78 | 1.44-2.2 | <0.0001 | 1.25 | 1.01-1.56 | 0.0445 | 1.06 | 0.84-1.32 | 0.6338 |
| Diabetes | 2.89 | 2.14-3.92 | <0.0001 | 2.17 | 1.6-2.95 | <0.0001 | 1.35 | 0.74-2.44 | <0.001 |
| Coronary artery disease | 3.36 | 2.82-4.67 | <0.0001 | 2.06 | 1.57-2.69 | <0.0001 | 1.68 | 1.27-2.22 | <0.001 |
| BMI, per Increase In 1 kg/m2 | 1.00 | 0.99-1.01 | 0.3489 | 1.01 | 1-1.02 | 0.0189 | 1.01 | 1.00-1.02 | 0.0375 |
| KK | 3.02 | 2.44-3.74 | <0.0001 | 1.96 | 1.54-2.5 | <0.0001 | 1.81 | 1.42-2.32 | <0.001 |

TABLE 12
KK genotype aggravated heart failure risk throughout 20-year follow-up for
individuals at high-risk, as well as for individuals considered at low risk.

| | Unadjusted | | | Age and Sex Adjusted | | | Multivariate Model | | |
|---|---|---|---|---|---|---|---|---|---|
| | Hazard | (95% | p- | Hazard | (95% | p- | Hazard | (95% | p- |
| | Ratio | CI) | value | Ratio | CI) | value | Ratio | CI) | value |
| | Incidence at 5 years | | | | | | | | |
| Age, per 1 year increase | 1.08 | 1.06-1.11 | <0.0001 | 1. [symbol] | 1.06-1.11 | <0.001 | 1.07 | 1.0 [symbol] -1.1 [symbol] | < [symbol] .001 |
| Men | 1.16 | [symbol] .74-1. [symbol] 3 | 0.5226 | 1.22 | 0.77-1.93 | 0.3692 | 0.9 [symbol] | 0. [symbol] 0-1.56 | 0.87 [symbol] |
| Hypertension | 1.55 | 0.98-2.44 | 0. [symbol] 16 | [symbol] | 0.6-1.54 | 0.8783 | 0.78 | 0.45-1.35 | 0.3742 |
| Diabetes | 4.46 | 2.62-7.58 | <0.0001 | 3.41 | 1.99-5.83 | <0.0001 | 2.75 | 1.57-4.8 [symbol] | <0.001 |
| Coronary artery disease | 5. [symbol] 8 | 3.57-9.03 | <0.0001 | 3.32 | 1.99-5.54 | <0.0001 | 2.69 | 1.53-4.72 | <0.001 |
| BMI, per Increase In 1 kg/m² | 1.01 | 8.99-1.03 | 0.3676 | 1.01 | 1-1.0 [symbol] | 0.0563 | 1.01 | 1-1. [symbol] 3 | 0.1358 |
| Presumed healthy + | | Referent [symbol] | | | Referent [symbol] | | | Referent [symbol] | |
| Not KK carrier | | | | | | | | | |
| Presumed low risk + | 4.56 | 1.61-12.93 | 0.0044 | 4.22 | 1.49-11.96 | 0.0069 | 4.33 | 3.62-12.28 | 0.00 [symbol] |
| KK carrier | | | | | | | | | |
| High risk subgroup + | 2.66 | 1.4-5. [symbol] 5 | 0.0027 | 1.81 | 0.95-3.47 | 0.0723 | 1.19 | 0.52-2.70 | 0. [symbol] 09 |
| Not KK carrier | | | | | | | | | |
| High risk subgroup + | 8.70 | 4.02-1 [symbol] .81 | <0.0001 | 5.84 | 2. [symbol] 8-12.72 | <0.0001 | 3.28 | 1.28-8.50 | 0.0147 |
| KK carrier | | | | | | | | | |
| | Incidence at 10 years | | | | | | | | |
| Age, per 1 year Increase | 1.08 | 1. [symbol] 6-1.09 | <0.0001 | 1. [symbol] 7 | 1.0 [symbol] -1.09 | <0. [symbol] 01 | 1.07 | 1.05-1.08 | <0.001 |
| Men | 1.0 [symbol] | 0.77-1.42 | 0.7 [symbol] 9 | 1. [symbol] 9 | 0. [symbol] -1.48 | 0.6891 | 0.91 | 0. [symbol] -1.2 [symbol] | 0.5 [symbol] |
| Hypertension | 1.79 | 1.32-2.43 | 0.0002 | 1.1 [symbol] | 0.85-1. [symbol] | 0.3535 | 0.77 | [symbol] .54-1.12 | 0.1765 |
| Diabetes | 3.41 | 2.3-5.07 | <0.0001 | 2.62 | 1.7 [symbol] -3.91 | <0.0001 | 1.95 | 1.29-2.95 | 0.001 [symbol] |
| Coronary artery disease | 4.26 | 3.06- [symbol] .93 | <0.0001 | 2. [symbol] 5 | 1.78-3.65 | <0.0 [symbol] 01 | 1. [symbol] 4 | 1.25-2.70 | 0.0019 |
| BMI, per Increase In 1 kg/m² | 1.01 | [symbol] .99-1.02 | 0.5221 | 1.01 | 1-1.02 | 0.0 [symbol] 32 | 1.01 | 0.9 [symbol] -1.02 | 0.5 [symbol] 12 |
| Presumed healthy + | | Referent [symbol] | | | Referent [symbol] | | | Referent [symbol] | |
| Not KK carrier | | | | | | | | | |
| Presumed low risk + | 8.60 | 4.31-17.16 | <0.0001 | 7.92 | 3.97-15.81 | <0.0001 | 8.03 | 4.02-16.02 | <0.001 |
| KK carrier | | | | | | | | | |
| High risk subgroup + | 4.39 | 2.6 [symbol] -7.15 | <0.0001 | 3.19 | 1.95-5.23 | <0.0001 | 2.85 | 1.59- [symbol] .12 | <0.001 |
| Not KK carrier | | | | | | | | | |
| High risk subgroup + | 10.47 | [symbol] .74-1 [symbol] .12 | <0.0001 | 7.57 | 4.1 [symbol] -13.88 | <0.0001 | [symbol] .24 | 3.13-12.43 | <0.001 |
| KK carrier | | | | | | | | | |
| | Incidence at 20 years | | | | | | | | |
| Age, per 1 year Increase | 1.08 | 1.06-1.09 | <0.0001 | 1.07 | 1. [symbol] -1.08 | <0.0001 | 1.07 | 1.06-1.0 [symbol] | <0.001 |
| Men | 1.25 | 1.01-1.54 | 0.0421 | 1.34 | 1.08-1.65 | 0.0075 | 1.20 | 0.96-1.49 | 0.1109 |
| Hypertension | 1.76 | 1.44-2.2 | <0.0001 | 1.25 | 1.01-1.56 | 0.0445 | 0.93 | 0.71-1.22 | 0.8144 |
| Diabetes | 2.89 | 2.14-3.92 | <0.0001 | 2.17 | 1.6-2.95 | <0.0001 | 1.79 | 1.30-2.45 | <0.001 |
| Coronary artery disease | 3.63 | 2.62- [symbol] .67 | <0.0001 | 2.0 [symbol] | 1.57-2.69 | <0.0001 | 1.65 | 1.23-2.19 | <0.001 |
| BMI, per Increase In 1 kg/m² | 1.00 | 0.99-1.01 | 0.3489 | 1.01 | 1-1.02 | 0.01 [symbol] 9 | 1.01 | 1-1.02 | 0.2376 |
| Presumed healthy + | | Referent [symbol] | | | Referent [symbol] | | | Referent [symbol] | |
| Not KK carrier | | | | | | | | | |
| Presumed low risk + | 3.97 | 2.4 [symbol] -5.38 | <0.0001 | 3.6 | 2.23-5.79 | <0.0001 | 3.62 | 2.25-5.83 | <0.001 |
| KK carrier | | | | | | | | | |
| High risk subgroup + | 2.42 | 1.85-3.16 | <0.0001 | 1.88 | 1.44-2.47 | <0.0001 | 1.57 | 1.10-2.23 | 0.012 [symbol] |
| Not KK carrier | | | | | | | | | |
| High risk subgroup + | 6.19 | 4.27- [symbol] .97 | <0.0001 | 4.7 | 3.23-6.82 | <0.0001 | 3.88 | 2.51-5.98 | <0.001 |
| KK carrier | | | | | | | | | |

[symbol]

indicates data missing or illegible when filed

TABLE 13
KK genotype aggravates heart failure risk throughout 20-year follow-up for
individuals with elevated, as well as individuals with normal NTproBNP.

| | Unadjusted | | | Age and Sex Adjusted | | | Multivariate Model | | |
|---|---|---|---|---|---|---|---|---|---|
| | Hazard | (95% | p- | Hazard | (95% | p- | Hazard | (95% | p- |
| | Ratio | CI) | value | Ratio | CI) | value | Ratio | CI) | value |
| | Incidence at 5 years | | | | | | | | |
| Age, per 1 year increase | 1. [symbol] 8 | 1.06-1.11 | <0.0001 | 1. [symbol] 8 | 1.06-1.11 | <0.001 | 1.05 | 1. [symbol] 3-1.08 | <0.001 |
| Men | 1.1 [symbol] | 0.73-1.63 | 0.527 [symbol] | 1.29 | 0.82-2. [symbol] 8 | 0.2768 | 1.16 | 0.72-1.89 | 0. [symbol] 411 |
| Hypertension | 1.54 | 0.97-2.43 | 0.0646 | [symbol] .96 | 0.6-1.54 | 0.8759 | 0.75 | 0.43-1.13 | 0.1465 |
| Diabetes | 4.44 | 2.61-7.56 | <0.0001 | 3.41 | 1.99-5.63 | <0.0001 | 2.80 | 1. [symbol] 1-4.86 | <0.001 |
| Coronary artery disease | 5.66 | 3.58-9 | <0.0001 | 3.32 | 1.99-5.54 | <0.0001 | 2.30 | 1.36-3.87 | 0.001 [symbol] |
| BMI, per Increase In 1 kg/m² | 1.01 | 0.98-1.03 | 0.3613 | 1.01 | 1-1.03 | 0.0562 | 1.02 | 1-1.04 | 0.0578 |
| Normal NT-proBNP-Not XK carrier | | Referent [symbol] | | | Referent [symbol] | | | Referent [symbol] | |
| Normal NT-proBNP- KK carrier | 1.00 | 1.43-6.2 [symbol] | [symbol] .0026 | 1.00 | 1.43-5.26 | 0.0025 | 2.44 | 1.26-4.76 | 0.0069 |
| Elevated NT-proBNP-Not KK carrier | 1.27 | 1.41-9.09 | [symbol] .0075 | 1.2 [symbol] | 0.83-5.8 [symbol] | 0.1121 | 1.85 | 6.6 [symbol] -4.76 | 0.2185 |
| Elevated NT-proBNP-KK carrier | 2.85 | 8.33-33.33 | <0.0001 | 0.42 | 4.78-25 | <0.0001 | [symbol] .33 | 3.70-20 | <0.001 |
| | Incidence at 10 years | | | | | | | | |
| Age, per 1 year Increase | 1.0 [symbol] | 1.06-1.09 | <0.0001 | 1.54 | 1.03-1.07 | <0.0001 | 1.06 | 1.04-1.07 | <0.001 |
| Men | 1.05 | [symbol] .77-1.42 | 0.7781 | 1. [symbol] 4 | 1.24-1.92 | <0.001 | 1.10 | 0.79-1.52 | 0. [symbol] 743 |
| Hypertension | 1.7 [symbol] | 1.31-2.42 | 0.0002 | 1.16 | 0.85-1.59 | 0.3564 | 0.96 | 0.69-1.32 | 0.7655 |
| Diabetes | 3.4 | 2.29-5.05 | <0.0001 | 2.62 | 1.75-3.91 | <0.0001 | 2.19 | 1.45-3.29 | <0.001 |
| Coronary artery disease | 4.24 | 3.04-5.91 | <0.0001 | 2.55 | 1.78-8.50 | <0.0001 | 1. [symbol] 0 | 1.31-2.75 | <0.001 |
| BMI, per Increase In 1 kg/m² | 1.01 | 0.99-1.02 | 0. [symbol] 069 | 1.01 | 1.78-8. [symbol] | 0.0 [symbol] 29 | 1.01 | 1-1.03 | 0.0922 |
| Normal NT-proBNP-Not XK carrier | | Referent [symbol] | | | Referent [symbol] | | | Referent [symbol] | |
| Normal NT-proBNP- KK carrier | 1.01 | 1. [symbol] 9-4.35 | <0.0001 | 2.40 | 1.35-3.13 | <0.001 | 2.63 | 1. [symbol] 1-4.35 | <0.001 |
| Elevated NT-proBNP-Not KK carrier | 1.09 | 1.56-5.56 | 0.0008 | 1.25 | 0.76-2.04 | 0.366 | 1.67 | 1.61-4.35 | 0.125 |
| Elevated NT-proBNP-KK carrier | 2.44 | 6. [symbol] 7-20 | <0.0001 | 4.35 | 2.86-6.87 | <0.001 | 5.88 | 3.45-10 | <0.001 |
| | Incidence at 20 years | | | | | | | | |
| Age, per 1 year Increase | 1.08 | 1. [symbol] -1.09 | <0.0001 | 1.0 [symbol] | 1.07-1.08 | <0.001 | 1.06 | 1.05-1.07 | <0.001 |
| Men | 1.24 | 1.01-1.54 | 0.0438 | 1.38 | 1.12-1.71 | 0.0028 | 1.38 [symbol] | 1.08-1.70 | 0.0078 |
| Hypertension | 1.77 | 1.43-2.19 | <0.0001 | 1.25 | 1-1. [symbol] | 0.0456 | 1.06 | 0. [symbol] -1. [symbol] | 0.5674 |
| Diabetes | 2. [symbol] | 2.13-3.9 | <0.0001 | 2. [symbol] 7 | 1.8-2.94 | <0.0001 | 1.95 | 1.43-2.66 | <0.001 |
| Coronary artery disease | 3. [symbol] 2 | 2.61-4. [symbol] 5 | <0.0001 | 2.0 [symbol] | 1.57-2.68 | <0.0001 | 1. [symbol] 5 | 1.25-2.18 | <0.001 |
| BMI, per Increase In 1 kg/m² | 1. [symbol] | 1-1.01 | 0.3343 | 1.01 | 1-1.02 | 0.01 [symbol] 7 | 1.01 | 1.00-1. [symbol] | 0.0232 |
| Normal NT-proBNP-Not XK carrier | | Referent [symbol] | | | Referent [symbol] | | | Referent [symbol] | |
| Normal NT-proBNP- KK carrier | 2.04 | 1.33- [symbol] .03 | 0.0009 | 2.04 | 1.35-3.1 | 0.000 [symbol] | 2.00 | 1.33-3.03 | 0.001 |
| Elevated NT-proBNP-Not KK carrier | 1.81 | 1.11-2.94 | 0.0177 | 1.25 | 0.76-2. [symbol] 4 | 0.3 [symbol] 6 | 1.12 | [symbol] 8-1.85 | 0. [symbol] 375 |
| Elevated NT-proBNP-KK carrier | 8.67 | 4.35-10 | <0.0001 | 4.35 | 2.86- [symbol] .87 | <0.0001 | 4.00 | 2.5 [symbol] -6.25 | <0.001 |

[symbol]

indicates data missing or illegible when filed

TABLE 14
Hazard ratios for the KK genotype across risk
factors for heart failure at 20-years.

| | Unadjusted | | | Age and Sex Adjusted | | |
|---|---|---|---|---|---|---|
| | Hazard | (95% | p- | Hazard | (95% | p- |
| | Ratio | CI) | value | Ratio | CI) | value |
| Age >65 | 1.56 | 1.05-2.33 | 0.0293 | 1.76 | 1.18-2.63 | 0.0059 |
| Sex | | | | | | |
| Women | 3.81 | 2.64-5.49 | <0.0001 | 2.06 | 1.38-3.09 | 0.0005 |
| Men | 2.36 | 1.57-3.53 | <0.0001 | 3.92 | 2.72-5.65 | <0.0001 |
| No | 3.23 | 2.25-4.64 | <0.0001 | 2.93 | 2.04-4.21 | <0.0001 |
| Hyper- | | | | | | |
| tension | | | | | | |
| Hyper- | 2.65 | 1.77-3.97 | <0.0001 | 2.59 | 1.73-3.88 | <0.0001 |
| tension | | | | | | |
| No | 3.09 | 2.31-4.13 | <0.0001 | 2.63 | 2.12-3.79 | <0.0001 |
| Diabetes | | | | | | |
| Diabetes | 1.94 | 1.04-4.02 | <0.0062 | 2.35 | 1.12-4.93 | 0.0240 |
| No | 1.15 | 0.57-2.31 | <0.6898 | 1.20 | 0.60-2.42 | 0.6092 |
| Coronary | | | | | | |
| artery | | | | | | |
| disease | | | | | | |
| Coronary | 3.59 | 2.68-4.82 | <0.0001 | 3.41 | 2.54-4.58 | <0.0001 |
| artery | | | | | | |
| disease | | | | | | |
| BMI <30 | 2.85 | 2.02-4.03 | <0.0001 | 2.66 | 1.88-3.76 | <0.0001 |
| BMI >/30 | 3.22 | 2.10-4.96 | <0.0001 | 3.33 | 2.16-5.13 | <0.0001 |
| Low Risk | 4.11 | 2.55-6.63 | <0.0001 | 3.64 | 2.28-6.87 | <0.0001 |
| subgroup | | | | | | |
| High Risk | 2.52 | 1.82-3.50 | <0.0001 | 2.44 | 1.76-3.39 | <0.0001 |
| subgroup | | | | | | |

These results demonstrate that E23K genotyping can enrich heart failure screening. For example, a E23K genotype can be used to identify a mammal as being at no or at lower risk of heart failure, while a KK status can be used as a theranostic marker for heart failure prediction and management.

### Example 4: Asymmetric Heart Failure Risk Imposed By E23k Genotype: Protective Heterozygocity Informs Personalized Therapeutic Strategy

KK homozygotes for the E23K variant of KCNJ11-encoded Kir6.2 constituting the pore subunit of ATP-sensitive K⁺ (K_{ATP}) channels can be at high risk of developing heart failure. In contrast, EE homozygotes and EK heterozygotes are at significantly lower risk. See, e.g., Examples 1-3. This Example evaluates genotype-imposed asymmetric considering that Kir6.2 exists as a multimeric ensemble.

Allelic frequency, deduced from a 2031-large community population, delineated E versus K stoichiometry in Kir6.2 tetramer assembly. In individuals carrying both E and K alleles, 99% of all Kir6.2 channels expressed at least one E-containing subunit. The disease resistant status of EK, across the 20-year follow-up in the tested community, informed that one E23 residue within the Kir6.2 tetramer is necessary and sufficient to avoid vulnerability. Leveraging this innate adaptive configuration can establish a translational prospect for converting KK homozygotes into a resilient phenotype that assures at least a single K residue substitution with an E counterpart in the Kir6.2 tetramer. In this way, a disease modifier paradigm that achieves proactive rectification of compromised cardioprotective K_{ATP} channels would offer a strategy fit to reduce individual heart failure risk.

### Beyond Conventional Allele-Based Dominant and Recessive Mutations

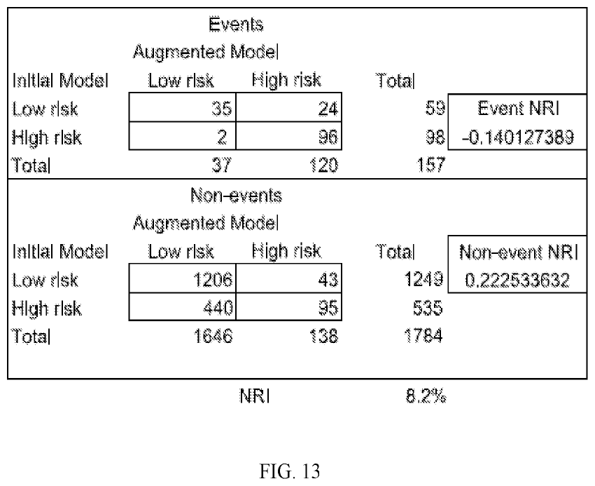

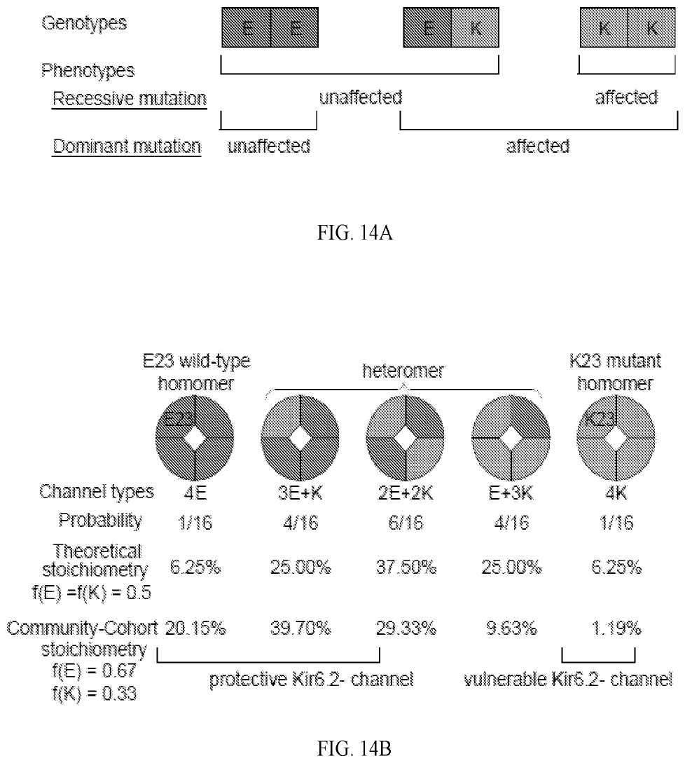

In homozygosity, an individual carries the identical inherited DNA sequence for a particular gene from both parents. In heterozygosity, an individual carries different forms of a specific gene from each respective parent. Recessive mutation implicates homozygosity for mutant alleles needed to express a patho-phenotypic pattern. As two copies of the defective gene are required to develop disease, heterozygous carriers are unaffected. In contrast, dominant mutations produce a disease phenotype not only in mutant homozygotes but also in heterozygous individuals that carry one mutant and one normal allele, as only one copy of the defective gene can trigger disease manifestation (FIG. 14A). The conventional one-to-one relationship assumes a gene-to-protein-to-pathophenotype. However, this simplified assumption may prove to be an oversimplification, especially for multimeric protein structures.

The pore-forming subunit of ATP-sensitive potassium (K_{ATP}) channels, namely the Kir6.2 protein, is formed by tetrameric assembly. Kir6.2 E23K polymorphism implicates that both EE homozygous and EK heterozygous genotypes carry significantly lower heart failure risk compared to KK mutant homozygous, implying a recessive mutation based on traditional interpretation. Beyond equivalent hetero-multimer allocation, and due to probabilistic permutations, heart failure-resilient EK heterozygous carriers can in principle produce a departure from a normal distribution of wildtype versus pathogenic mutant channels. An assortment of multimeric options therefore invites further considerations beyond the conventional allele-based dominant and recessive mutation models.

### Tetrameric Kir6.2 Structural Assembly Predicts Assortment of Channel Subtypes

Individuals carrying Kir6.2 EE or KK homozygosity produce homo-tetrameric Kir6.2 channel comprising either 4E wildtype or 4K mutant, respectively. EK heterozygotes, however, display a variety of multimeric channel options imposed by the tetrameric rather than monomeric organization of the Kir6.2 pore.

Assuming that the Kir6.2 subunit assembly is unbiased, independent and random, a binomial distribution can be expected. Accordingly, the theoretical frequencies (P(x)) of Kir6.2 channel pore subtypes were computed:

$$P ( x ) = ( \begin{matrix}n \\ x\end{matrix} ) * {p}^{x} * {q}^{n - x} = \frac{n !}{( n - x ) ! * x !} * {p}^{x} * {q}^{n - x}$$

where n=4 as the Kir6.2 channel is a tetramer, x is the number (0-4) of E-containing subunits (or (4−x) K-containing subunits) in a particular assembled channel, p and q (q=1−p) are the allelic probability of E or K being incorporated in any of four possible channel subunit positions.

Assuming that in a heterozygote the allelic probability of E and K is equal to 0.5, enabling random and independent co-assembly of E- and K-containing subunits, a binomial distribution predicted 16 different channel subtype options, grouped into 1/16 with no mutant subunits (4E), 4/16 with one mutant subunit (3E+K), 6/16 with two mutant subunits (2E+2K), 4/16 with three mutant subunits (E+3K), and 1/16 with 4 mutant subunits (4K). In this way, 87.50% channels were predicted to contain heteromeric channel subtypes with a mixture of E and K (3E+K, 2E+2K, E+3K) (FIG. 14B). Moreover, the likelihood that each homo-tetrameric wildtype (4E) and mutant (4K) channel subtype is expressed was 6.25%. Distinctive from traditional dominant/recessive models, where heteromers would yield 50% of wildtype and 50% of mutant proteins, tetrameric Kir6.2 channel assembly provokes a distinguishing asymmetry generating an assortment of channel assembly options.

EK Heterozygosity Implies that 99% of Kir6.2 Channels Carry ≥1 E-Containing Subunit in the Population

Surveillance of a community, comprised of 2031 individuals, computed the real-world E/K allelic frequencies. Based on the actual genotype distribution in the population, namely 885, 960, and 186 for EE, EK, and KK, respectively, the allelic frequency of E and K was:

Allele frequency for E, f(E)=p=[2×obs(EE)+obs(EK)]/[2×(obs(EE+EK+KK)]

Allele frequency for K, f(K)=q=[2×obs(KK)+obs(EK)]/[2×(obs(EE+EK+KK)]

where obs(EE), (EK), and (KK) are number of individuals genotyped for EE, EK or KK, respectively.

Community cohort-imposed allelic frequency of E and K were 0.67 and 0.33 respectively, unmasking an unequal distribution of E predominating over K. The calculated frequency in this community was validated for the Kir6.2 E23K polymorphism across populations of East Asian, European and Hispanic, excluding African American with significantly lower K allelic frequency (0.03-0.07; see, e.g., broadcvdi.org/variantInfo/variantInfo/rs5219).

The actual permutation of possible Kir6.2 channel subtypes in EK heterozygosity produced 16 different channel subtype options, with however different probability outcomes. The higher allelic frequency of E forecasted a substantially increased homo-tetrameric wildtype channel subtypes (4E; 20.15%), and a significantly decreased home-tetrameric mutant channel subtypes (4K; 1.19%). The heteromeric channel subtypes with a combination of E and K were 78.66%, a relative reduction from an equal allelic frequency calculation (FIG. 14B). Remarkably, this community cohort-based assessment indicated that the cardioprotective EK heterozygous carrier produced 99% of the channel harboring at least one E-containing subunit in the assembled tetrameric Kir6.2 channel.

### Restoring Innate Protective Kir6.2 Configuration

Resilient heterozygotes displayed 99% of K_{ATP} channels with at least one wildtype E23 residue within the Kir6.2 tetramer, suggesting that one E23 subunit per assembled pore is necessary and sufficient to overcome cardioprotective vulnerability. This is consistent with the notion that although each Kir6.2 subunit possesses an ATP binding site, adenine nucleotide binding to a single Kir6.2 subunit per the assembled pore tetramer is sufficient for channel closure. Accordingly, the innate cardioprotective Kir6.2 configuration relying on one E23-containing subunit per assembled pore offers a translational prospect of converting vulnerable KK homozygotes into resilient EK heterozygotes though finite K-to-E replacement.

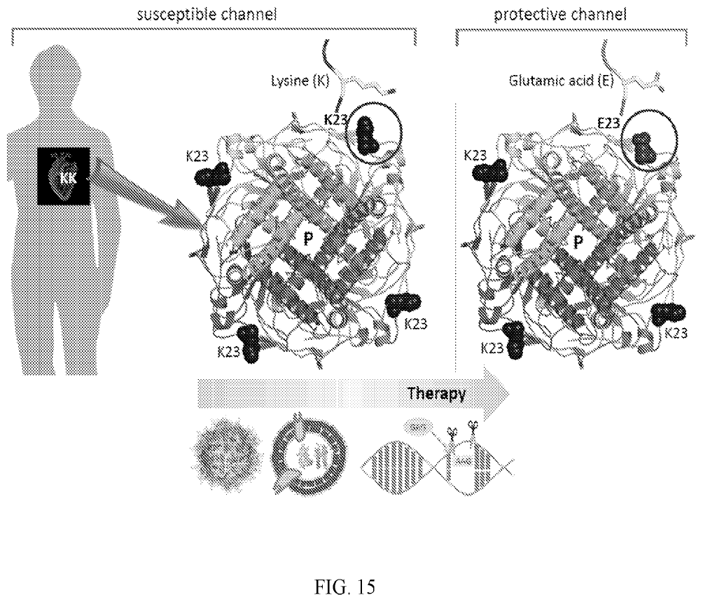

Together, these results demonstrate that, viral and non-viral gene therapy approaches along with gene editing modalities can be used to treat mammal's at high risk of developing heart failure (FIG. 15). For example, mammals having genome that is homozygous for the c.67G>A single nucleotide variant in a KCNJ11 gene encoding a Kir6.2-E23K (KK) polypeptide can have an increased risk of developing heart failure, and can be treated using a gene therapy or gene-editing approach to convert at least one c.67G>A single nucleotide variant in a KCNJ11 gene such that mammal is can express a Kir6.2 K_{ATP} channel pore having at least one E-containing (E23) subunit.

### Example 5: Pathogenic KIR6.2 E23K Polymorphism Violates K_{ATP} Channel Interaction

To address mutation-induced structural alteration and define the basis for vulnerability associated with the E23K variant, molecular bioinformatics were here applied. The iterative approach delineated a E23 enabled electrostatic path in support of Kir6.2-Kir6.2 and Kir6.2-SUR communication, disrupted by the pathogenic K23 counterpart. This deep learning-aided roadmap offers a means to predict outcome caused by a genetic variance.

### Materials and Methods

### In Silico Single Nucleotide Polymorphism Prediction

To predict phenotypic outcome(s) associated with the non-synonymous E23K mutation in the human KCNJ11-encoded Kir6.2 pore protein, a computational surveillance algorithm for single nucleotide polymorphism (SNP) screening (PredictSNP; loschmidt.chemi.muni.cz/predictsnp/) was applied. The PredictSNP tool integrates multiple parameters deduced from evolutionary information, physico-chemical characteristics or structural traits, and employs machine learning—based on training datasets of annotated mutations—to derive a graded score ranging from ‘neutral’ to ‘deleterious’. Notably, the PredictSNP dataset archives more than forty thousand mutations facilitating unbiased SNP evaluation. Use of the consensus classifier PredictSNP program was here amplified with additional SNP prediction tools. Namely, the multivariate analysis of protein polymorphism (MAPP), the predictor of human deleterious single nucleotide polymorphism (PhD-SNP), the polymorphism phenotyping-1 and 2 (PolyPhen-1 and 2), the sorting intolerant from tolerant (SIFT), the screening for non-acceptable polymorphism (SNAP), and the protein analysis through evolutionary relationships (PANTHER) were all employed to enhance prediction performance.

### Deep Learning Protein Structural Analytics

To decipher intricate structural features of the human Kir6.2 protein, an integrated deep learning system (NetSurfP-2.0; cbs.dtu.dk/services/NetSurfP/) was utilized. NetSurfP-2.0 computes the protein complex architecture constructed from convolutional and long short-term memory neural networks, trained on solved protein structures to refine the protein disorder prediction output. To fortify the accuracy of prediction, the NetSurfP-2.0 deep neural network program leveraged a pre-established benchmark, namely the DisProt database a resource comprising experimentally annotated disordered proteins.

### Iterative Molecular Threading

To build a reliable molecular model of human Kir6.2, including the E23 containing amino-terminal region, the I-TASSER (Iterative Threading ASSEmbly Refinement) platform was used (zhanglab.ccmb.med.umich.edu/I-TASSER/). I-TASSER employs a hierarchical approach to protein structure based on the sequence-to-structure-to-function paradigm. From the starting amino acid sequence, implementation of multiple threading alignments and iterative structural assembly refinement simulations generated 3-dimensional atomic structure model options. The I-TASSER program covered comparative modeling to ab initio folding, providing accuracy and reliability for the full-length structure. Assembly of Kir6.2 monomers into a tetrameric complex was built using the reliability of the geometry-based docking program Symmdock, and best fit was selected according to shape complementarity. Protein structures were visualized using Pymol (pymol.org/2/), a molecular visualization computer software system.

### Protein Sequence Alignment

To assess evolutionary conservation, amino acids alignment options were digitized using a high-quality configuration program (Clustal Omega; ebi.ac.uk/Tools/msa/clustalo/). This bioinformatics program uses guided trees, and hidden Markov model profile-profile techniques, to generate sequence alignment scenarios across species. Of note, multiple sequence alignment with Clustal Omega here outperformed in accuracy and quality curated alternatives, including a Benchmark alignment database (BaliBase), a protein reference alignment benchmark (Prefab), and a fully automated and highly scalable benchmark (QuanTest).

### Protein-Protein Docking

To delineate electrostatic interactions of human Kir6.2 with regulatory SUR2A proteins, the high ambiguity driven biomolecular docking program (HADDOCK; haddock.science.uu.nl/services/HADDOCK2.2/) was employed. HADDOCK has demonstrated reliable accuracy of prediction. Indeed, HADDOCK uses structural conformation-based flexible docking to build a biomolecular complex model using the encoded information from identified or predicted protein interfaces in ambiguous interaction restraints to drive the docking process. The scoring function here guided the continuous conformation space search until the interaction restraints were satisfied. To this end, the human Kir6.2 subunit was docked to the recently resolved SUR2A-equivalent human SUR1 (PDB ID: 6C3O). Formation of the biomolecular Kir6.2-SUR complex was tested under the restraints of Kir6.2 K338 and SUR1 D1354 (equivalent to SUR2A E1318). These residues were pre-identified by mutational permutation as poles underlying electrostatic Kir6.2-SUR interaction.

### Molecular Dynamic Simulations

The molecular behavior of E23- versus K23-containing Kir6.2 tetramers in solution was assessed using the biomolecular dynamics simulation software Gromacs (gromacs.org/) replicating Newtonian equations of motion. Atomic coordinate versus time was derived under a 54a7_lipid force field. Specifically, the Kir6.2 tetramer was embedded in a membrane lipid bilayer, constructed with 330 1,2-dipalmitoyl-sn-glycero-3-phosphocholine (DPPC) molecules with a 40.6 Å bilayer thickness. The aqueous environment was simulated with an explicit simple point-charge water model in a triclinic system with a minimum water thickness of 20 Å at both sides of the lipid bilayer. Positive charges were neutralized with Cl⁻, and 0.1 M sodium chloride was introduced to mimic physiological conditions. Kir6.2 tetramers were subjected to iterative descent energy minimization with a maximum force of 300 kJ·mol⁻¹·nm⁻². System equilibration was performed under constant volume and temperature (NVT) which equilibrated the system to 310 K with a velocity-rescaling thermostat for 200 ps, and under constant temperature and pressure (NPT) which equilibrated the system to 1.0 bar at 310 K with the Berendsen weak coupling method for 600 ps. Heavy atom position restraints were used during equilibration of the NVT/NPT ensemble. Molecular dynamics simulations employed periodic boundary conditions with an integration time step of 3.0 fs. Long-range electrostatic interactions were calculated using the particle mesh Ewald method with a Fourier grid spacing of 0.12 nm, and the LINCS algorithm was adopted to control for bond lengths. Trajectories of molecular dynamics simulations were recorded at every 30 ps.

### Results

### Disordered Kir6.2 E23 Region

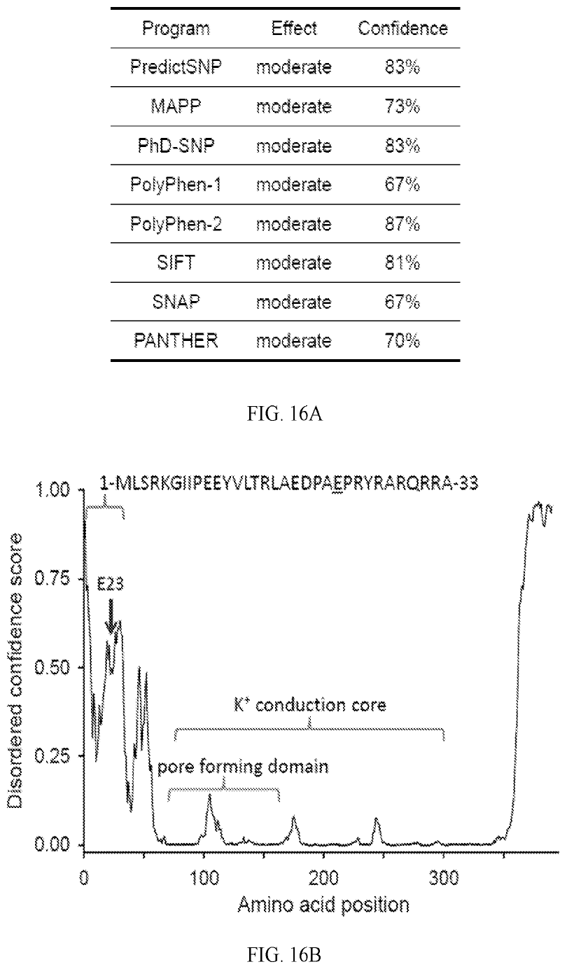

Multimodal computational tools were here used to assess the E23K mutation in the Kir6.2 K_{ATP} channel pore. Single nucleotide polymorphism (SNP) survey projected a non-destructive, moderate effect from the E to K non-synonymous mutation (FIG. 16A), contradicting the pathogenic outcome associated with disease risk epidemiologically documented for homozygous E23K (KK) carriers. As initial SNP inference failed to map the impact of the clinically relevant E23K missense mutation, a deep learning protein disorder algorithm was then applied in order to delineate the secondary Kir6.2 protein structure. Within the 390 amino acids constituting Kir6.2, the amino terminal 1 through 33 residues—including E23—manifested a highly disordered confidence score, distinct from the ordered K⁺ conduction core including the pore forming domain (FIG. 16B). Predicting the impact of E23K thus remains uncertain, confounded by the intrinsically unstructured nature of the Kir6.2 encompassing E23 region.

### E23 Enabled Kir6.2-Kir6.2 Interactions

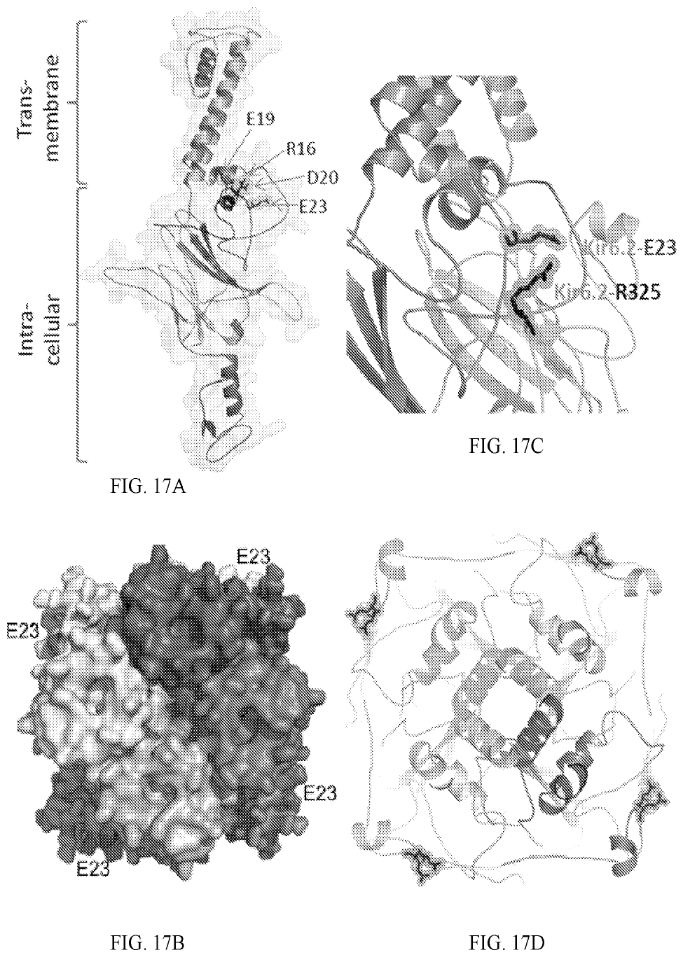

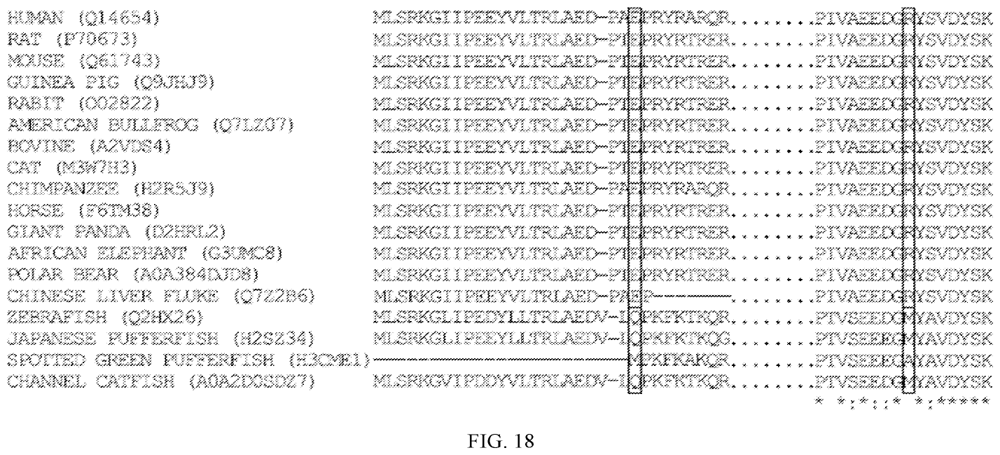

Unfolding the disordered E23-containing region leveraged an all-atom construction of the Kir6.2 protein. Atomistic assessment located the negatively charged E23 at the transmembrane/intracellular interface (FIG. 17A). Within the vicinity of plasma-membrane phospholipid head groups, the E23 locale clustered around charged R16, E19 and D20, yet the exposed E23 remained unengaged from direct interactions with adjacent residues (FIG. 17A). Rather, E23 mapped at the periphery of the four assembled Kir6.2 monomers away from the centrally situated channel pore (FIG. 17B). Within the -70 Å-large tetrameric structure, close-up evaluation revealed attraction of the negatively charged E23 with the positively charged R325 residing within a neighboring Kir6.2 monomer (FIGS. 17C & 17D). The favored E23/R325 ionic interaction formed a stabilizing electrostatic Kir6.2-Kir6.2 pairing, with evolutionary conservation validating the criticality of the pair (FIG. 18). Specifically, sequence alignment of Kir6.2 orthologs demonstrated preservation of the E23/R325 couple across mammals and non-mammals. In rare species where the negatively charged residue at position E23 was replaced with an uncharged alternative, the R325 counterpart remained also uncharged avoiding electrostatic repulsion (FIG. 18). Well-preserved structural conformity thus underpins a strategic ionic interaction supporting Kir6.2-Kir6.2 homomer communication.

### Conformationally Viable Kir6.2-SUR Heteromer

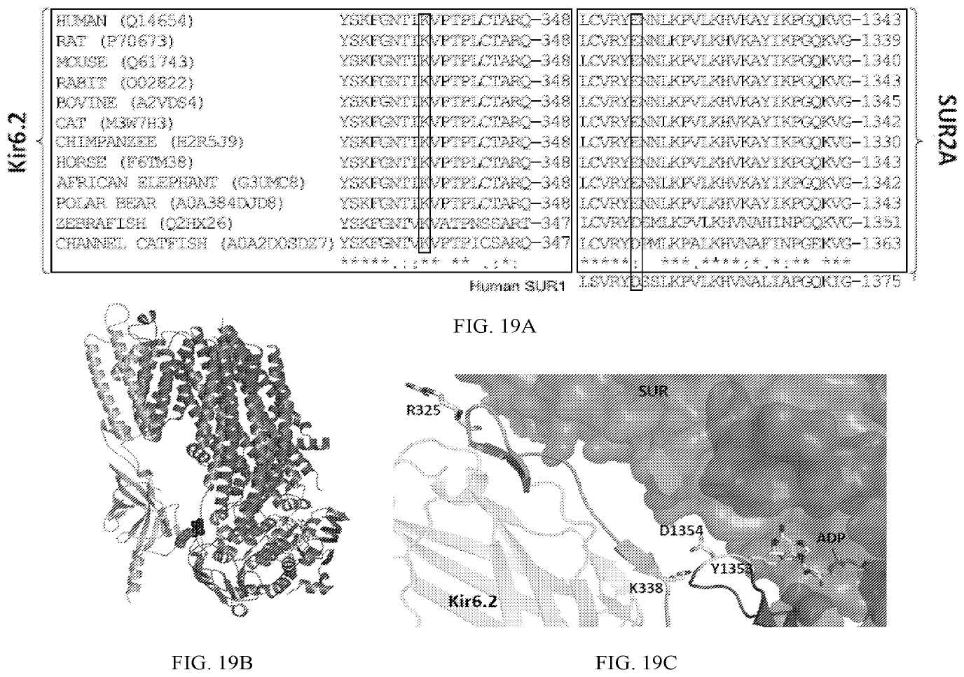

For heteromeric interaction with surrounding SUR regulatory proteins, the Kir6.2 forming pore relies on an exclusive salt bridge downstream of the E23/R325 pair, implicating K338 in Kir6.2 and E1322 in the SUR2A subunit (or the equivalent D1354 in the SUR1 homolog). This distinctive interaction was here documented to be conserved among species, and consistently formed by the positively charged K338 residue in Kir6.2 paired with a negatively charged counterpart in SUR (FIG. 19A). Restraint-based high ambiguity driven biomolecular docking deduced 166 possible conformational options in achieving a conformationally viable Kir6.2-SUR tandem. The most probable coordinated twin structure, resulting in the best scoring function, positioned Kir6.2 intimately within the SUR proximity (nearest distance: -3.2 Å between Kir6.2 P340 and SUR1 F1398 within the cytoplasmic locale). A rotated Kir6.2 embraced the cytoplasmic D1354-containing region of SUR. The salt bridge between Kir6.2 K338 and SUR D1354 was fortified by adjacent π-π stacking formed by the aromatic side chain of SUR Y1353 and the purine ring of SUR-bound ADP, stabilizing the Kir6.2 and nucleotide-bound SUR conformation (FIGS. 19B & 19C). This finding, deduced from the biomolecular complex model built with Kir6.2 and the SUR2A homolog SUR1, emphasizes the influence of E23 in ensuring the permissive electrostatic coupling pathway Kir6.2 E23/R325 followed by Kir6.2-K338/SUR2A-E1322, needed for stability and integrity of the Kr6.2-SUR multimeric complex.

### K23 Mutation Imposes a Repulsive Scenario

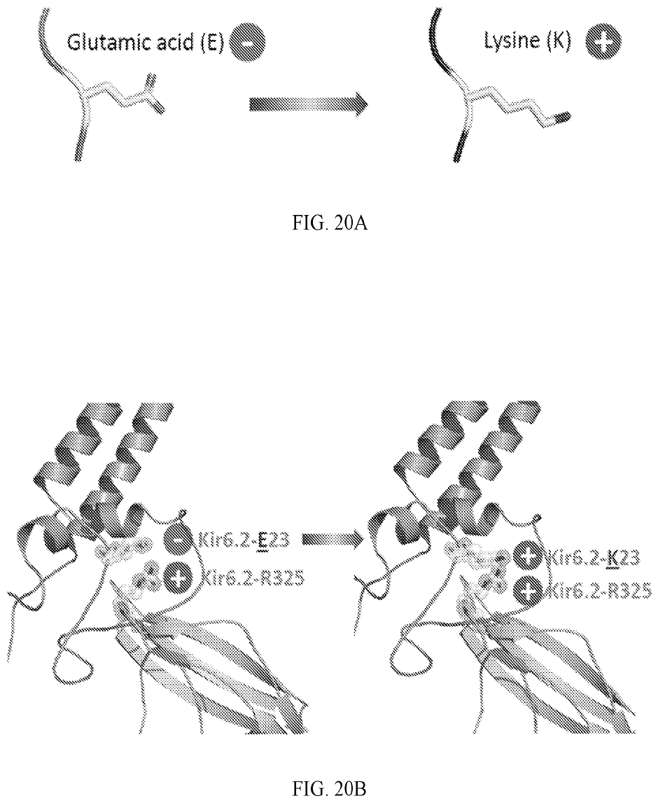

The switch from the highly conserved negatively charged E23 glutamic acid to the K23 variant introduced a bulkier and positively charged lysine residue (FIG. 20A).

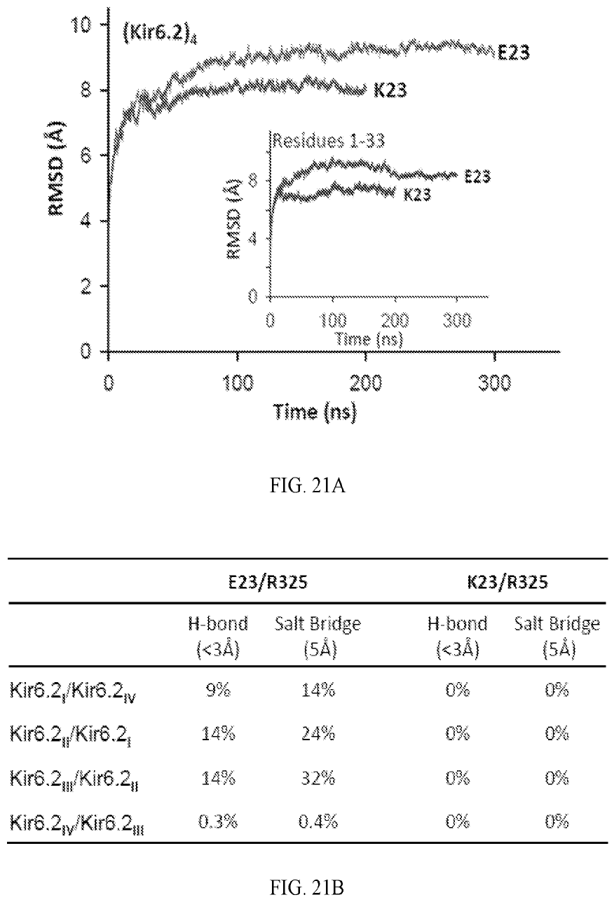

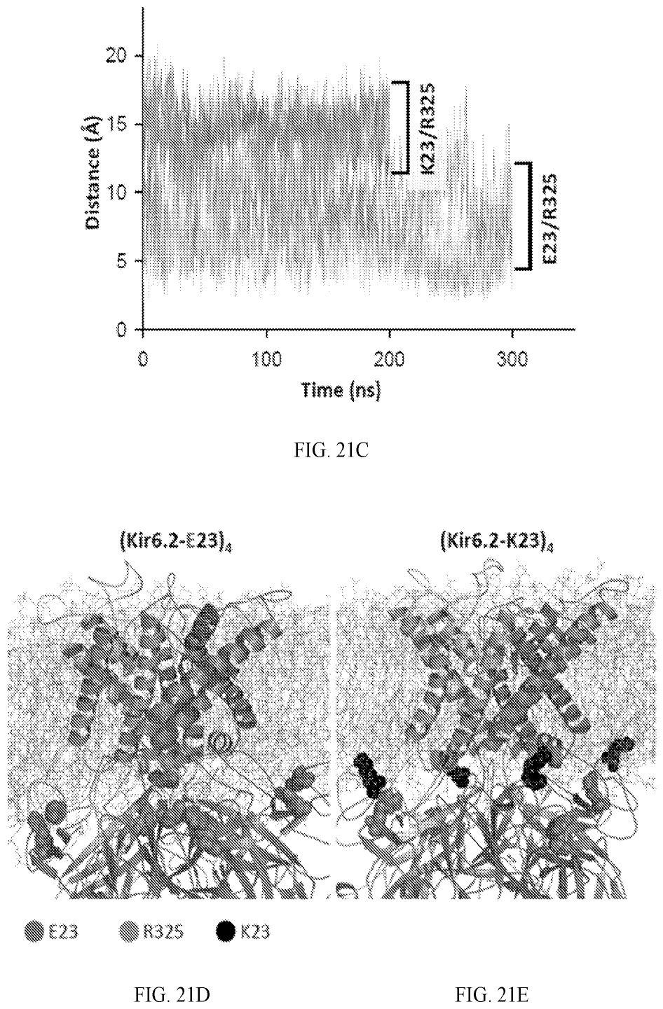

Accordingly, the conditions for electrostatic pairing normally occurring between E23 and R325 were ruptured by the oppositely charged K23, corrupting a permissive electrostatic setting (FIG. 20B). The ensuing repulsive scenario, introduced by the incompatible E23 to K23 transition, was thus evaluated by molecular dynamics simulation. Specifically, the molecular behavior of E23- versus K23-containing Kir6.2 tetramers was assessed in a lipid (1,2-dipalmitoyl-sn-glycero-3-phosphocholine) bilayer by hundreds nanosecond-long molecular dynamics simulations (FIG. 21A). In both E23 and K23 Kir6.2 tetramers, a significant conformational drift occurred within the first 90 ns of simulation, reaching a plateau of the root-mean-square deviation (RMSD), a measure of the average distance between a-carbon atoms between initial and simulated protein structures (FIG. 21A). Notably, the RMSD behavior distinguished K23 from E23 Kir6.2 tetramers (FIG. 21A), a mutation-based distinction reproduced within the mobile K23 versus E23 regions (FIG. 6A inset). In fact, K23 never achieved close contact with R325, contrasting with the realized proximity between the E23/R325 pair matching in three out of four configurations (FIG. 21). The probability of hydrogen bonding and salt-bridging was moreover higher in the setting of the E23/R325 interacting pair, separating from the repulsive K23/R325 duo that oscillated further away from the recognized ionic interaction standard (FIG. 21C). Contrasting E23/R325 ionic pairing, K23 was repulsed from R325 and attracted by the negatively charged lipid head (FIGS. 21D and 21E). Collectively, K23 introduces a non-permissive, repulsive scenario thereby destabilizing channel subunit interactions.

Together these results demonstrate that a clinically relevant Kir6.2 E23K polymorphism associated with heart failure can violate the permissive electrostatic pairing path compromising cardioprotective K_{ATP} channel subunit interactions.

### Example 6: Transgenic KK Carrier Animal Model Prone To Heart Failure

### Methods

A transgenic murine model was engineered to express the E23K (KK) polymorphism within the Kir6.2 ATP-sensitive K⁺ (K_{ATP}) channel pore. In short, cloning and recombination-mediated genetic engineering techniques were used to construct the targeting vector. Designed targeting vector encompassed long homology arm, Kcnj11 codon substitution GAG>AAG (E23K), neomycin selection cassette flanked with a flippase recognition target (FRT) sequence, and short homology arm. The linearized targeting vector was transfected by electroporation into FLP C57Bl/6 (BF1) embryonic stem cells. After antibiotic selection, surviving clones were expanded for PCR analysis to identify recombinant embryonic stem cell clones. Identified positive embryonic stem cells were micro-injected into Balb/c blastocysts, and resulting chimeras with a high percentage of black coat color were mated with C57BL/6 wild type mice to generate germline neomycin deleted mice. Mice that carry the Kcnj11 E23K mutation were confirmed by genotyping and sequencing. Age- and sex-matched heterozygote EK and homozygote KK animals underwent left nephrectomy followed by subcutaneous implantation of a desoxycorticosterone acetate (DOCA) pellet (150 mg, 60 days release) and were supplemented with high salt diet (1% NaCl and 0.2% KCl in drinking water) for 60 days post-surgery. Impact of DOCA-induced pro-hypertensive stress condition was prospectively evaluated by monitoring systemic heart failure syndrome parameters (survivorship, body weight, treadmill exercise test, and whole body metabolism), tail blood pressure, electrocardiography, high-resolution echocardiography, cardiac catheterization, and histopathology upon autopsy.

### Results

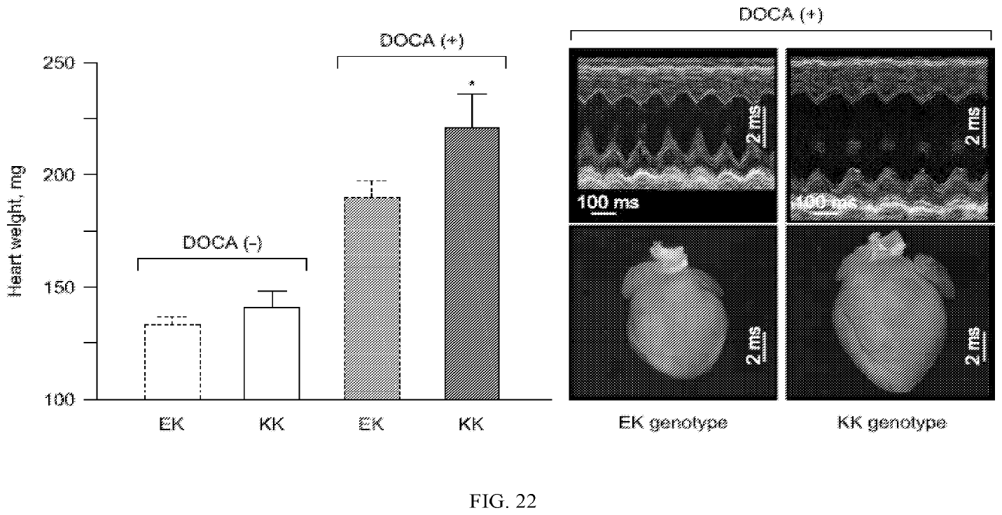

A murine model was genetically engineered to express the E23K (KK) polymorphism in the Kir6.2 ATP-sensitive K⁺ (K_{ATP}) channel pore. While indistinguishable at baseline from non-KK counterparts, namely EK littermates, KK homozygotes displayed pronounced vulnerability to develop heart failure (FIG. 22). Specifically, in the setting of imposed hemodynamic load (DOCA), KK hearts were found markedly enlarged on pathological examination, to a degree that significantly exceeded that of age/sex-matched EK heterozygotes (FIG. 22). Exaggerated cardiomegaly under stress in KK carriers was associated with reduced contractility revealed by echocardiography (FIG. 22). The engineered KK model thus exhibits, in the absence of potentially confounding variables, an increased heart failure risk. By recapitulating the innate myocardial susceptibility associated with the KK polymorphism in humans, this newly developed transgenic model offers a valuable platform to guide the further understanding of the mechanistic basis of disease predisposition and, in tandem, the development of targeted therapeutic solutions aimed to rescue heart failure prone carriers.

### Example 7: E23K Mutant Accumulates Heart Failure-Associated Branched-Chain Amino Acid Metabolites

This Example evaluates whether targeted rescue of metabolically compromised KK hearts can turn-on deficient cardiac BCAA catabolism to delay heart failure progression.

### Methods

High-resolution ¹H nuclear magnetic resonance spectroscopy (NMR) fingerprinted 31 metabolites in wild-type (EE) and transgenic E23K homozygous mutant (KK) hearts.

### Results

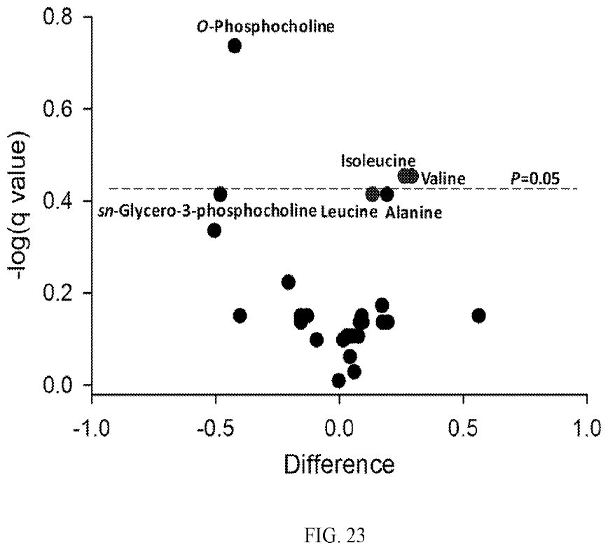

Volcano plot analysis revealed in KK, compared to EE counterparts, elevated O-phosphocholine, isoleucine, and valine (p<0.05), along with marginal change in leucine (p=0.055) (FIG. 23). Excess in branched-chain amino acids (BCAA) is a hallmark of a failing heart. This would suggest that KK carriers accumulate BCAAs in the context of a downregulated BCAA catabolism.

The results suggest that inhibitors of branched-chain a-ketoacid dehydrogenase kinase (BCKDK), such as 3,6-dichlorobenzothiophene-2-carboxylaic acid (BT2), can be used as a pharmacological intervention to treat heart failure.

### Example 8: Treating Heart Failure

Nucleic acid encoding a Kir6.2-E23 polypeptide is administered to cells within a human identified as having the presence of a c.67G>A single nucleotide variant in both copies of a KCNJ11 gene. The nucleic acid encoding a Kir6.2-E23 polypeptide is expressed by the cell such that the cells form functional K_{ATP} channels (e.g., K_{ATP} channels having at least one Kir6.2-E23 polypeptide).

The administered nucleic acid encoding a Kir6.2-E23 polypeptide can slow, delay, or reverse heart failure. For example, the administered nucleic acid encoding a Kir6.2-E23 polypeptide can reduce the severity of one or more symptoms of heart failure.

### Example 9: Treating Heart Failure

Kir6.2-E23 polypeptides are administered to cells within a human identified as having the presence of a c.67G>A single nucleotide variant in both copies of a KCNJ11 gene such that the cells form functional K_{ATP} channels (e.g., K_{ATP} channels having at least one Kir6.2-E23 polypeptide).

The administered Kir6.2-E23 polypeptides can slow, delay, or reverse heart failure. For example, the administered Kir6.2-E23 polypeptides can reduce the severity of one or more symptoms of heart failure.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-6. (canceled)

7. A method for treating a mammal at risk of developing heart failure, wherein said method comprises:
administering, to cells within a mammal identified as having a c.67G>A single nucleotide variant in both copies of a KCNJ11 gene of said mammal, nucleic acid encoding a Kir6.2-E23 polypeptide, wherein said Kir6.2-E23 polypeptide is expressed by said cells, and wherein said cells form ATP-sensitive potassium (K_{ATP}) channels having at least one Kir6.2-E23 polypeptide.

8. The method of claim 7, wherein said mammal is a human.

9. The method of claim 7, wherein said cells are cardiac cells.

10. The method of claim 7, wherein said Kir6.2-E23 polypeptide is a human Kir6.2-E23 polypeptide.

11. The method of claim 7, wherein said nucleic acid encoding said Kir6.2-E23 polypeptide is administered to said cells in the form of a viral vector.

12. The method of claim 11, wherein said viral vector is selected from the group consisting of adeno-associated viral vectors, Sendai viral vectors, lentiviral vectors, retroviral vectors, adenoviral vectors, and herpes simplex viral vectors.

13. The method of claim 7, wherein said nucleic acid encoding said Kir6.2-E23 polypeptide is administered to said cells in the form of a non-viral vector.

14. The method of claim 13, wherein said non-viral vector is selected from the group consisting of extracellular vesicles, liposomes, and expression plasmids.

15. The method of claim 7, wherein said nucleic acid encoding said Kir6.2-E23 polypeptide is operably linked to a promoter sequence.

16. The method of claim 7, wherein said administration of said nucleic acid encoding a Kir6.2-E23 polypeptide is selected from the group consisting of an intracoronary injection, an endomyocardial injection, an epicardial injection, a coronary sinus injection, and a pericardial injection.

17-18. (canceled)

19. A method for treating a mammal having heart failure, wherein said method comprises:
administering, to cells within a mammal identified as having a c.67G>A single nucleotide variant in both copies of a KCNJ11 gene of said mammal, nucleic acid encoding a Kir6.2-E23 polypeptide, wherein said Kir6.2-E23 polypeptide is expressed by said cells, and wherein said cells form ATP-sensitive potassium (K_{ATP}) channels having at least one Kir6.2-E23 polypeptide.

20. The method of claim 19, wherein said mammal is a human.

21. The method of claim 19, wherein said cells are cardiac cells.

22. The method of claim 19, wherein said Kir6.2-E23 polypeptide is a human Kir6.2-E23 polypeptide.

23. The method of claim 19, wherein said nucleic acid encoding said Kir6.2-E23 polypeptide is administered to said cells in the form of a viral vector.

24. The method of claim 23, wherein said viral vector is selected from the group consisting of adeno-associated viral vectors, Sendai viral vectors, lentiviral vectors, retroviral vectors, adenoviral vectors, and herpes simplex viral vectors.

25. The method of claim 19, wherein said nucleic acid encoding said Kir6.2-E23 polypeptide is administered to said cells in the form of a non-viral vector.

26. The method of claim 25, wherein said non-viral vector is selected from the group consisting of extracellular vesicles, liposomes, and expression plasmids.

27. The method of claim 19, wherein said nucleic acid encoding said Kir6.2-E23 polypeptide is operably linked to a promoter sequence.

28. The method of claim 19, wherein said administration of said nucleic acid encoding a Kir6.2-E23 polypeptide is selected from the group consisting of an intracoronary injection, an endomyocardial injection, an epicardial injection, a coronary sinus injection, and a pericardial injection.

29-30. (canceled)
